(19) United States
(12) Patent Application Publication
(10) Pub. No.: US 2012/0321609 A1
(43) Pub. Date: Dec. 20, 2012

(54) METHOD OF TREATING PSYCHOLOGICAL DISORDERS

(76) Inventors: Stephen Ferguson, London (CA); Ana Magalhaes, London (CA); Hymie Anisman, Manotick (CA)

(21) Appl. No.: 13/444,481

(22) Filed: Apr. 11, 2012

Related U.S. Application Data
(60) Provisional application No. 61/473,855, filed on Apr. 11, 2011.

(51) Int. Cl. A61K 38/08; A61K 38/46; A61K 31/711; G01N 33/566; A61P 25/22; A61P 25/24; G01N 33/573; A61K 31/351; A61K 38/10

(52) U.S. Cl. 424/94.6; 514/460; 514/44 R; 514/21.6; 514/21.5; 514/17.5; 514/17.6; 435/7.4; 435/7.21

(57) ABSTRACT

A method of desensitizing 5-HT₂R signaling in the mammal is provided. The method comprises one or more of:
1) inhibiting CRFR1 activation of 5-HT_{2A}R signaling by preventing trafficking of intracellular vesicles or blocking recycling of 5-HT_{2A}R to the cell surface;
2) blocking PDZ binding motifs in the carboxyl-terminal tail domains of at least one of CRFR1, 5-HT_{2A}R or 5-HT_{2C}R; or
3) blocking the interaction of a 5-HT₂R or a CRFR1 with a PDZ-domain-containing protein selected from the group consisting of MAGI-1 PDZ1, MAGI-2 PDZ1, MAGI-3 PDZ1, PSD95 PDZ 1&2, PSD95 PDZ3, CAL PDZ, SAP97 PDZ 1&2, PTPN13 PDZ 4&5, PDZK2 PDZ1, MPP3 PDZ, ERBIN PDZ and MUPP1 PDZ 12.

This application claims the benefit of U.S. Provisional Patent Application No. 61/473,855, filed on Apr. 11, 2011, and incorporates such provisional patent application in its entirety by reference.

### FIELD OF THE INVENTION

The present invention relates to a method of treating psychological disorders, and more particularly, to a method of modulating the interaction between a corticotropin releasing factor receptor and a serotonin receptor.

### BACKGROUND OF THE INVENTION

Anxiety and major depressive disorder often present as co-morbid disorders and the expression and severity of these disorders is commonly associated with stressful experiences. In response to stress, corticotropin releasing factor (CRF) regulates the activity of hypothalamic-pituitary-adrenal (HPA) axis and triggers changes in other neurotransmitters systems, such as serotonin (5-HT). CRF is also known to influence anxiety responses and CRF receptor 1 (CRFR1) may be particularly important in this regard. 5-HT also has diverse functional effects in the central nervous system, as well as in the periphery and plays an important role in modulating depression and anxiety-related behaviours in humans and rodents. In particular, pharmacological studies and knockout mice have demonstrated that 5-HT_{2A} and 5-HT_{2C} receptors contribute to anxiety and are pharmacological targets for the treatment of anxiety. The targeted deletion of either the 5-HT_{2A}R, 5-HT_{2C}R or CRFR1 in mice is also associated with a reduction in anxiety-related behaviour. However, little is known about the molecular mechanisms underlying the cross talk between these two important neurotransmitter systems at the cellular level.

CRF is a 41 amino acid peptide that activates the HPA axis to regulate adrenocorticotropin secretion by the pituitary gland in response to acute and chronic stress. CRF peptide acts through two subtypes of Gs-coupled G protein-coupled receptors (GPCRs) resulting in increased intracellular cAMP formation. Besides its endocrine function in the pituitary, CRF is also involved in a wide variety of effects not related to its pituitary activity indicating it also functions as either a neurotransmitter or neuromodulator in the brain. Consistent with its role as a neurotransmitter, CRF immunoreactive terminals, CRF binding sites and CRF receptor mRNA are widely distributed in areas of the brain that are unrelated to endocrine function. There are also fifteen genes encoding functional serotonin receptors (5-HTR) in the mammalian brain that are classified into 7 families (5-HT₁ to 5-HT₇), all of which are GPCRs except for 5-HT₃Rs which are ionotropic receptors.

The 5-HT₂ and CRF receptors each contribute to the regulation of anxiety behaviors and stress responses and CRF treatment is demonstrated to prolong 5-HT regulation of GABAergic inhibitory transmission. The molecular and cellular basis for the action of CRF on 5-HT signaling remains unknown, as agents that increase cAMP accumulation do not mimic the effect of CRFR activation.

It would be desirable, thus, to develop methods of regulating 5-HT₂ or CRF receptors that would be useful in the treatment of psychological disorders such as anxiety and related behaviors.

### SUMMARY OF THE INVENTION

The present invention relates to methods of desensitizing 5-HT₂R signaling which are useful to treat psychological disorders.

In one aspect, a method of desensitizing 5-HT₂R signaling is provided and comprises inhibiting CRFR1 activation of 5-HT_{2A}R signaling by preventing trafficking of intracellular vesicles or endocytosis.

In another aspect, a method of desensitizing 5-HT₂R signaling is provided and comprises inhibiting CRFR1 activation of 5-HT_{2A}R signaling by blocking recycling of 5-HT_{2A}R to the cell surface.

In another aspect, a method of desensitizing 5-HT₂R signaling is provided and comprises blocking PDZ binding motifs in at least one of CRFR1, 5-HT_{2A}R and 5-HT_{2C}R.

In a further aspect, a method of desensitizing 5-HT₂R signaling is provided and comprises blocking the interaction of a 5-HT₂R or CRFR1 with a PDZ-domain-containing protein.

These and other aspects are described in the detailed description that follows by reference to the following figures.

### BRIEF DESCRIPTION OF THE FIGURES

FIG. 1. Effect of CRFR1 activation on 5-HT₂R signaling. Dose response curves for 5-HT-stimulated inositol phosphate (IP) formation in HEK 293 cells pretreated with and without CRF (500 nM) for 30 min in cells transfected with (a) either FLAG-5-HT_{2A}R and FLAG-5-HT_{2c}R alone, (b) FLAG-5-HT_{2A}R and HA-CRFR1, or (c) FLAG-5-HT_{2c}R and HA-CRFR1. (d) Basal and agonist-stimulated inositol formation in cells expressing FLAG-5-HT_{2c}R alone, HA-CRFR1 alone, or expressing both FLAG-5-HT_{2c}R and HA-CRFR1. Cells were treated with 500 nM CRF with or without a subsequent exposure to 10 μM 5-HT for 30 min. (e) Dose response curves for 5-HT stimulated inositol phosphate formation in HEK 293 cells transfected with FLAG-5-HT_{2A}R and β₂AR and pretreated with and without 100 μM isoproterenol (Iso) for 30 min. (f) Dose response curves for 5-HT stimulated inositol phosphate formation in HEK 293 cells transfected with FLAG-5-HT_{2A}R and CRFR2 and pretreated with and without 500 nM CRF for 30 min. (g) Dose response curves for CRF-stimulated cAMP formation in HEK 293 cells transfected with FLAG-5-HT_{2A}R and HA-CRFR1 and pretreated with and without 10 μM 5-HT for 30 min. The data represent the mean±S.E.M. for 3-6 individual experiments.

FIG. 2. Role of endocytosis in CRFR1-dependent augmentation of 5-HT₂R signaling. (a) Dose response curves for 5-HT stimulated inositol phosphate (IP) formation in HEK 293 cells transfected with FLAG-5-HT_{2A}R and HA-CRFR1 and pretreated with and without 500 nM CRF for 30 min in the presence of dominant-negative dynamin I-K44A. The dose response curves represent the mean±S.E.M. for 4 independent experiments.

FIG. 3. Role of receptor recycling in CRF modulated 5-HT₂R signaling. (a) Dose response curves for 5-HT stimulated inositol phosphate (IP) formation in HEK 293 cells transfected with FLAG-5-HT_{2A}R and HA-CRFR1 and pretreated with and without 500 nM CRF for 30 min following the pretreatment of cells with and without 100 μM monensin for 30 min. (b) Dose response curves for 5-HT stimulated inositol phosphate formation in HEK 293 cells with transfected FLAG-5-HT_{2A}R, HA-CRFR1 and Rab4S8N and pretreated with and without 500 nM CRF for 30 min. (c) Dose response curves for 5-HT stimulated inositol phosphate formation in HEK 293 cells transfected with FLAG-5-HT_{2A}R, HA-CRFR1 and Rab11-S25N and pretreated with and without 500 nM CRF for 30 min. (d) Increase in cell surface 5-HT_{2A}R localization following 30 min pretreatment of CRFR1 with 500 nM CRF. The cell surface expression of the 5-HT_{2A}R represents the mean±S.E.M. for 4 independent experiments. * P<0.05 versus untreated control.

FIG. 4. Receptor determinants of CRF-dependent increases in 5-HT₂R signaling. (a) Shown is the change in cell surface 5-HT_{2A}R and 5-HT_{2A}R-ΔSCV localization following 30 min pretreatment of CRFR1 with 500 nM CRF as well as the change in cell surface 5-HT_{2A}R localization following 30 min pretreatment of CRFR1-ΔTAV with 500 nM CRF. The cell surface expression of the 5-HT_{2A}R represents the mean±S.E.M. for 4 independent experiments. *P<0.05 versus untreated control. (b) Dose response curves for 5-HT stimulated inositol phosphate (IP) formation in HEK 293 cells transfected with FLAG-5-HT_{2c}R and either HA-CRFR1 or HA-CRFR1 lacking a PDZ domain binding motif (ΔTAV) pretreated with and without 500 nM CRF for 30 min. (c) Dose response curves for 5-HT stimulated inositol phosphate formation in HEK 293 cells transfected with HA-CRFR1 and either FLAG-5-HT_{2C}R or FLAG-5-HT_{2C}R lacking a PDZ domain binding motif (ΔSSV) pretreated with and without 500 nM CRF for 30 min. (d) Dose response curves for 5-HT stimulated inositol phosphate formation in HEK 293 cells transfected with HA-CRFR1 and either FLAG-5-HT_{2A}R or FLAG-5-HT_{2A}R lacking a PDZ domain binding motif (ΔSCV) pretreated with and without 500 nM CRF for 30 min. (e) Dose response curves for 5-HT stimulated inositol phosphate formation in HEK 293 cells transfected with HA-CRFR1 and FLAG-5-HT_{2A}R pretreated for 1 h with a Tat-fusion peptide corresponding to the last 10 amino acid residues of the CRFR1 carboxyl-terminal tail and then treated with and without 500 nM CRF for 30 min. Dose response curves represent the mean±S.E.M. for 3-5 independent experiments.

FIG. 5. Analysis of CRF pretreatment on 5-HT₂R-mediated anxiety-related behaviours. (a) Mean latencies for mice to enter the center square in a 5 min open field. (b) Mean latency to enter the open arms of the elevated plus maze. (c) The frequency of entries in to the open arms of the elevated plus maze. (d) The frequency of entries in to the closed arms of the elevated plus maze. (e) Time spent in the closed arms of the elevated plus maze. In all experiments, either vehicle or CRF (1.5 μg in 1 μl) was administered to the medial prefrontal cortex via a surgically implanted cannulae for 5 min and 5 min later mice were intraperotineally injected with vehicle or DOI (0.15 mg/kg) prior to behavioral testing. 9-10 mice were used in each test group. P<0.01 versus vehicle/vehicle treated control. Data represents mean±SD. *P<0.01 versus vehicle/vehicle treated control.

FIG. 6. Analysis of CRF pretreatment on 5-HT₂R-mediated anxiety-related behaviours following M100907 treatment. (a) Mean latency to enter the open arms of the elevated plus maze in a 5 min test period. (b) The frequency of entries in to the open arms of the elevated plus maze. (c) Time spent in the open arms of the elevated plus maze. (d) The frequency of entries in to the closed arms of the elevated plus maze. (e) Time spent in the closed arms of the elevated plus maze. In all experiments, either vehicle or CRF (1.5 μg in 1 μl) was administered to the medial prefrontal cortex via a surgically implanted cannulae for 5 min and 5 min later mice were intraperotineally injected with vehicle or DOI (0.15 mg/kg) and mice were pretreated i.p. with either vehicle or 0.25 mg/kg of M100907 in a volume of 0.3 ml prior to DOI administration before behavioral testing. 6-8 mice were used in each test group. Data represents mean±SD. *P<0.05 versus respective vehicle control. ** P<0.05 versus respective M100907 treatment. ^{φ}P<0.05 relative to M100907 and CRF treatment.

FIG. 7. The CRFR1-CT binds to a specific subset of PDZ proteins. Equal amounts of purified His-tagged fusion proteins corresponding to a subset of PDZ domains were spotted on nylon membranes and overlay with GST (A) was compared with GST-CRFR1-CT (B) to reveal CRFR1-CT binding to the spotted PDZ domains. (C) Identity of 96 distinct PDZ domains that were spotted on nylon membranes. Data are representative of four independent experiments.

FIG. 8. YFP-SAP97 co-immunoprecipitates with HA-CRFR1 in the PDZ-binding motif-dependent, CRF agonist-independent manner. (A) Representative immunoblot of SAP97 co-immunoprecipitated with either HA-CRFR1 or not HA-CRFR1ΔTAV. Transient transfections were performed in HEK293 cells as labelled. Samples were run using SDS-PAGE and immunoblotted with rabbit anti-GFP. GFP-SAP97 co-immunoprecipitated with HA-CRFR1, but not HA-CRFR1ΔTAV which lacks the PDZ-binding motif. (B) Effect of CRF treatment was quantified using densitometry and had no significant effect on the amount of GFP-SAP97 co-immunoprecipitated with HA-CRFR1.

FIG. 9. SAP97 antagonizes HA-CRFR1 endocytosis. (A) Agonist-stimulated internalization of either HA-CRFR1 or HA-CRFR1-TAV in cells co-transfected with either GFP or GFP-SAP97. The internalization of HA tagged receptors labelled with Alexa Fluor-conjugated mouse anti-NA antibody was measured in cells treated with 500 nM CRF for 30 min and compared with vehicle treated control cells. The data represents the Mean±SEM of X independent experiments. * P<0.05 versus control CRFR1 internalization. (B) Representative immunoblot of endogenous SAP97 protein expression in HEK 293 cells transfected with 3 ug plasmid cDNA encoding either scrambled (SCR) or SAP97 shRNA at 48 and 72 h initial transfection. (C) Agonist stimulated (500 nM CRF) internalization of HA-CRFR1 in cells co-transfected with scrambled (SCR) and SAP shRNA at 0, 5, 15, 30 and 60 min. The data represents the Mean±SEM of X independent experiments. * P<0.05 versus SCR shRNA treated cells.

FIG. 10. SAP97 does not regulate CRFR1-mediated cAMP formation. (A) CRFR1-mediated cAMP formation, as assessed by a cAMP GLO assay, following co-transfection with either GFP (control) or GFP-SAP97. The data represents the Mean±SEM of X independent experiments. (B) CRFR1- and CRFR1-ΔTAV-mediated cAMP formation, as assessed by a cAMP GLO assay. The data represents the Mean±SEM of X independent experiments. (C) CRFR1-mediated-cAMP formation as assessed by a cAMP GLO assay following co-transfection with either scrambled (SCR) or SAP97 shRNA. The data represents the Mean±SEM of X independent experiments.

FIG. 11. Knockdown of endogenous SAP97 suppresses HA-CRFR1-mediated ERK1/2 phosphorylation. (A) Representative immunoblot showing SAP97 shRNA-mediated knockdown of endogenous SAP97 protein expression in HEK 293 cells. (B) Representative immunoblot showing ERK1/2 phosphorylation in response 500 nM CRF treatment for 0, 5, 15 and 30 min in non-transfected (NT) HEK 293 cell, and HEK 293 cells transfected with HA-CRFR1 and either scrambled (SCR) or SAP97 shRNA. (C) Densitometric analysis of ERK1/2 phosphorylation in response 500 nM CRF treatment for 0, 5, 15 and 30 min in non-transfected (NT) HEK 293 cell, and HEK 293 cells transfected with HA-CRFR1 and either scrambled (SCR) or SAP97 shRNA. The data represents the Mean±SEM of X independent experiments. * P<0.05 versus SCR shRNA treated cells.

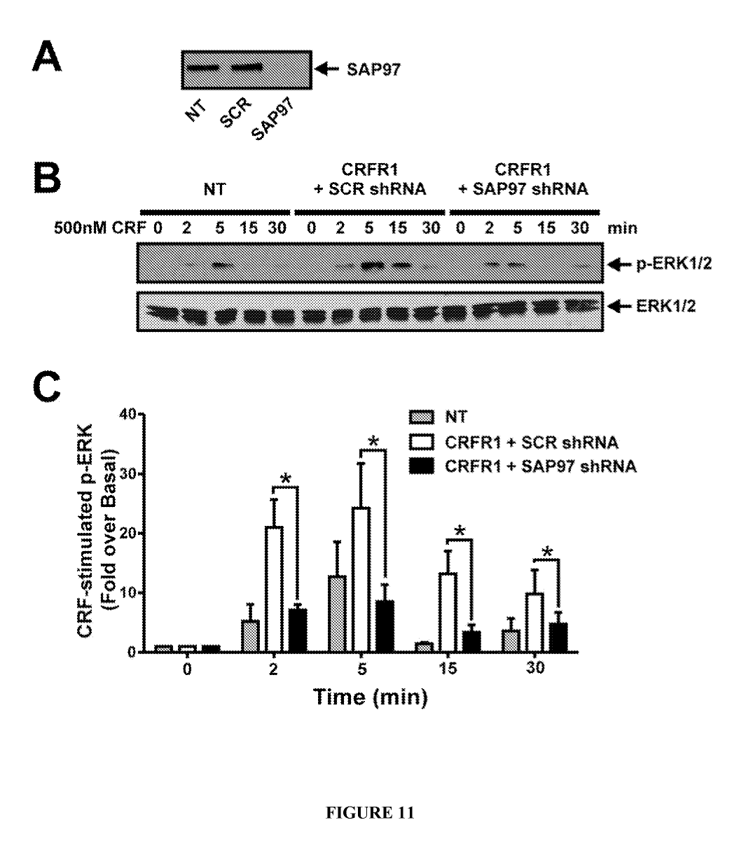

FIG. 12 illustrates the amino acid sequences of CRFR-1 (A), 5HT_{2A}R (B) and 5HT_{2C}R (C).

FIG. 13 illustrates the amino acid sequence of SAP97.

### DETAILED DESCRIPTION OF THE INVENTION

The present invention relates to methods of desensitizing 5-HT₂R signaling in a mammal. Such methods have been determined to be useful to treat psychological disorders in mammals.

The term “psychological disorder” includes anxiety-related disorders, depression and related disorders and stress-related disorders. Such disorders may include depression, schizophrenia, anxiety disorder, and bipolar disorder.

The term “mammal” is used herein to refer to either human or non-human mammals.

In a first aspect, 5-HT₂R signaling may be desensitized to treat a psychological disorder by inhibiting CRFR1 activation of 5-HT_{2A}R signaling. This may be achieved by administration of an agent that prevents trafficking of intracellular vesicles or endocytosis. In one embodiment, monensin was used to prevent such trafficking.

Desensitization of 5-HT₂R signaling may also be achieved by blocking rapid recycling of GPCRs to the cell surface. In one embodiment, overexpression of a dominant-negative Rab GTPase that selectively prevents receptor recycling, such as Rab4-S28N mutant protein, was useful to block recycling of 5-HT_{2A}R.

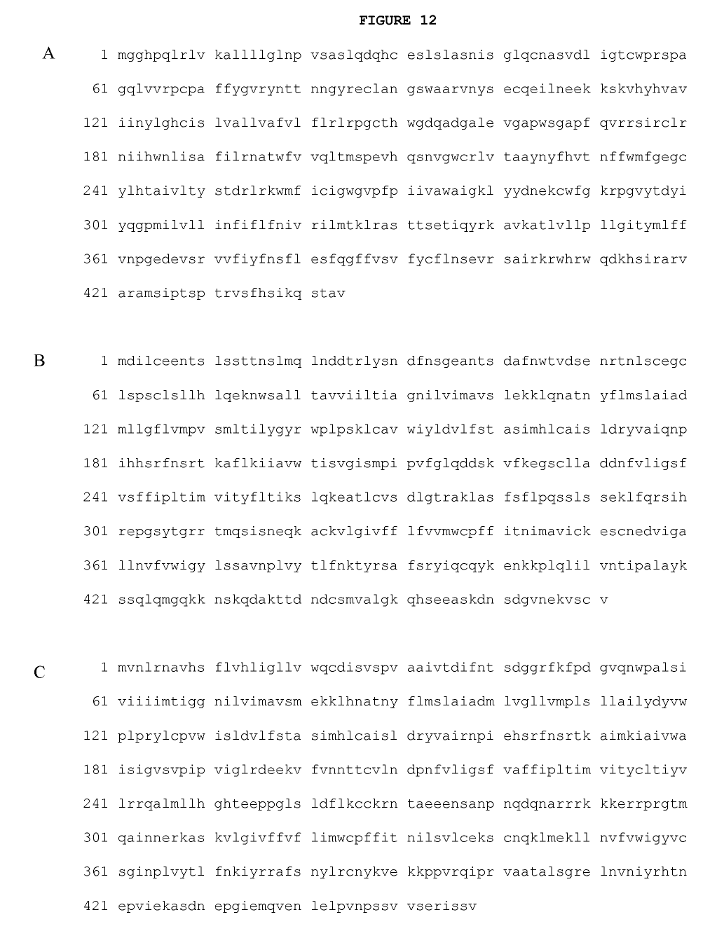

In another aspect, PDZ binding motifs in the carboxyl-terminal tail domains of at least one of CRFR1, 5-HT_{2A}R or 5-HT_{2C}R may be blocked to desensitize 5-HT₂R signaling in a mammal. The amino acid sequences of CRFR1, 5-HT_{2A}R or 5-HT_{2C}R, including the PDZ binding motifs of each, are set out in FIG. 12. PDZ binding motifs may be blocked using a variety of techniques. In one embodiment, peptides designed to block the PDZ binding motif may be used, e.g. peptides which mimic the natural ligand. An example of a blocking peptide for the CRFR1 PDZ binding motif may incorporate the C-terminal consensus sequence of this motif, e.g. including the STAV sequence. Examples of such blocking peptides include the 10 amino acid peptide, FHSIKQSTAV, TRVSFHSIKQSTAV, and functionally equivalent derivatives thereof, while an example of a blocking peptide for the 5-HT_{2A}R PDZ binding motif is KDNSDGVNEKVSCV, or functionally equivalent derivatives thereof. Functionally equivalent derivatives are derivatives of the peptide which retain activity to block the PDZ binding motif and include one or more amino acid substitutions such as conservative amino acid substitutions, e.g. leucine, alanine, isoleucine and valine may be substituted for one another, serine and threonine may be substituted for one another, arginine and lysine may be substituted for one another, asparagines and glutamine may be substituted for one another, and aspartic and glutamic acid may be substituted for one another, amino acid deletions or additions, or other modifications such as side chain modifications that do affect the blocking activity thereof. Peptide mimetics may also be prepared, for example, based on known peptide inhibitors. Such peptide mimetics may be designed to incorporate desirable features such as protease resistance. Generally, such peptide mimetics are designed based on techniques well-established in the art, including computer modelling.

In a further aspect, a method of desensitizing 5-HT₂R signaling is provided in which a PDZ-domain-containing protein is blocked from binding to the PDZ binding motif on a target 5-HT_{2A}R or CRFR1 in the mammal. PDZ-domain-containing proteins that may be blocked include, but are not limited to, MAGI-1 PDZ1, MAGI-2 PDZ1, MAGI-3 PDZ1, PSD95 PDZ 1&2, PSD95 PDZ3, CAL PDZ, SAP97 PDZ 1&2, PTPN13 PDZ 4&5, PDZK2 PDZ1, MPP3 PDZ, ERBIN PDZ and MUPP1 PDZ 12. Techniques known in the art may be used to block PDZ-domain-containing proteins, and thereby desensitize 5-HT₂R signaling.

Inhibitors that may be useful to block PDZ-domain-containing proteins include immunological inhibitors such as antibodies, e.g. polyclonal or monoclonal antibodies. Antibodies may be prepared using known methods. Monoclonal antibodies are prepared using the well-established hybridoma technology developed by Kohler and Milstein (Nature 256, 495-497 (1975)). Hybridoma cells can be screened immunochemically for production of antibodies specifically reactive with a selected SOX9 region and the monoclonal antibodies can be isolated. The term “antibody” as used herein is intended to include fragments thereof which also specifically react with a SOX9 protein according to the invention, as well as chimeric antibody derivatives, i.e., antibody molecules resulting from the combination of a variable non-human animal peptide region and a constant human peptide region.

Methods which prevent expression of the PDZ-domain proteins may also be employed including the use of antisense oligonucleotides, and RNA interfering nucleotides, e.g. siRNA, shRNA and miRNA. The term “antisense oligonucleotide” as used herein means a nucleotide sequence that is complementary to at least a portion of a target PDZ-domain protein-encoding nucleic acid sequence. The term “oligonucleotide” refers to an oligomer or polymer of nucleotide or nucleoside monomers consisting of naturally occurring bases, sugars, and intersugar (backbone) linkages. The term includes modified or substituted oligomers comprising non-naturally occurring monomers or portions thereof, which function similarly. The term also includes chimeric oligonucleotides which contain two or more chemically distinct regions. The antisense oligonucleotides of the present invention may be ribonucleic or deoxyribonucleic acids and may contain naturally occurring or modified bases. SiRNA technology may also be employed in which nucleic acid fragments such as siRNA fragments that correspond with regions of a gene encoding a PDZ-domain protein are used to block expression.

Antisense, siRNA, and other inhibitory nucleic acid molecules may be readily prepared using well-established methods of nucleic acid syntheses, given that the structure of target PDZ-domain proteins is known in the art. It will be appreciated by one of skill in the art that such inhibitory nucleic acids may be derived from specific regions of the target gene to provide more effective inhibition of gene expression, for example, the 5′ end of the gene. In addition, as one of skill in the art will appreciate, useful nucleic acid fragments may not correspond exactly with the target gene, but may incorporate sequence modifications, for example, addition, deletion or substitution of one or more of the nucleotide bases therein, provided that the modified siRNA retains the ability to bind to the target gene and block expression. Once prepared, oligonucleotides determined to be useful to inhibit gene expression, may be used to desensitize 5-HT₂R signalling. A suitable oligonucleotide may be introduced into tissues or cells of a mammal using techniques in the art including vectors (retroviral vectors, adenoviral vectors and DNA virus vectors) or by using physical techniques such as microinjection.

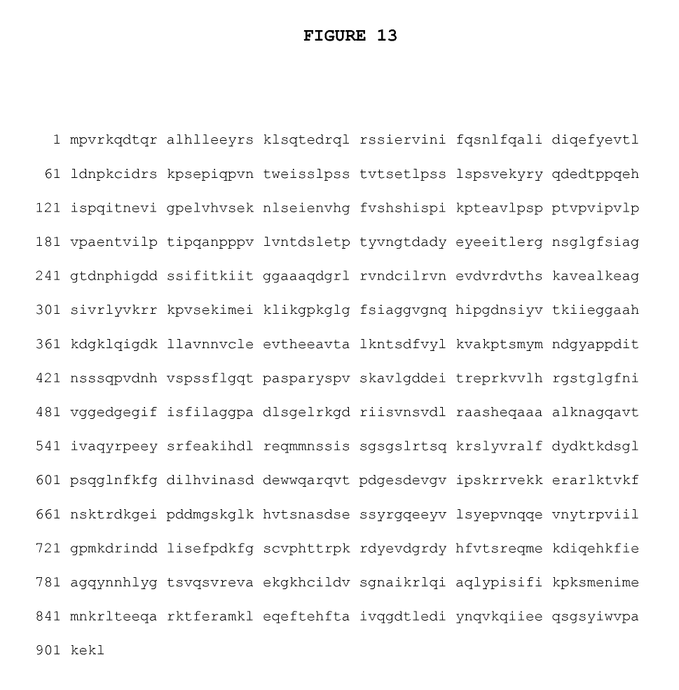

In one embodiment, the PDZ-domain-containing protein is the SAP97, or synapse-associated protein 97, which is also known as Disks large homolog 1 (DLG1). The amino acid sequence of this protein is set out in FIG. 13. This protein is shown to interact with the PDZ binding motif of CRFR1 and thereby regulate CRFR1 function. Accordingly, modulating the expression of SAP97, modulates CRFR1 function and 5-HT₂R signaling. SAP97 may be modulated using well-established techniques including, for example, immunological techniques (e.g. antibodies that target the PDZ domain of the SAP97 protein). Methods which prevent expression of SAP97 may also be employed including the use of antisense oligonucleotides, and RNA interfering nucleotides, e.g. siRNA, shRNA and miRNA, as described above.

An inhibitory agent determined to be useful to desensitize 5-HT₂R signaling may be administered to a mammal in need of treatment using any suitable mode of administration including, but not limited to, oral, subcutaneous, intravenous, intraperitoneal, intranasal, enteral, topical, sublingual, intramuscular, intra-arterial, intramedullary, intrathecal, inhalation, ocular, transdermal, vaginal or rectal means.

An inhibitory agent determined to be useful to desensitize 5-HT₂R signaling may be administered to a mammal in need of treatment alone or in combination with a suitable pharmaceutically acceptable carrier. The expression “pharmaceutically acceptable” means acceptable for use in the pharmaceutical and veterinary arts, i.e. not being unacceptably toxic or otherwise unsuitable. Examples of pharmaceutically acceptable carriers include diluents, excipients and the like. Reference may be made to “Remington's: The Science and Practice of Pharmacy”, 21st Ed., Lippincott Williams & Wilkins, 2005, for guidance on drug formulations generally. The selection of adjuvant depends on the type of inhibitor and the intended mode of administration of the composition. In one embodiment of the invention, the compounds are formulated for administration by infusion, or by injection either subcutaneously, intravenously, intrathecally, intraspinally or as part of an artificial matrix, and are accordingly utilized as aqueous solutions in sterile and pyrogen-free form and optionally buffered or made isotonic. Thus, the compounds may be administered in distilled water or, more desirably, in saline, phosphate-buffered saline or 5% dextrose solution. Compositions for oral administration via tablet, capsule or suspension are prepared using adjuvants including sugars, such as lactose, glucose and sucrose; starches such as corn starch and potato starch; cellulose and derivatives thereof, including sodium carboxymethylcellulose, ethylcellulose and cellulose acetates; powdered tragancanth; malt; gelatin; talc; stearic acids; magnesium stearate; calcium sulfate; vegetable oils, such as peanut oils, cotton seed oil, sesame oil, olive oil and corn oil; polyols such as propylene glycol, glycerine, sorbital, mannitol and polyethylene glycol; agar; alginic acids; water; isotonic saline and phosphate buffer solutions. Wetting agents, lubricants such as sodium lauryl sulfate, stabilizers, tableting agents, anti-oxidants, preservatives, colouring agents and flavouring agents may also be present. Aerosol formulations, for example, for nasal delivery, may also be prepared in which suitable propellant adjuvants are used. Other adjuvants may also be added to the composition regardless of how it is to be administered, for example, anti-microbial agents may be added to the composition to prevent microbial growth over prolonged storage periods.

In another aspect of the invention, SAP97 may be used as a tool to screen for therapeutic agents. Candidate inhibitors may be screened to identify compounds that alter the interaction between SAP97 and CRFR1 or 5HT₂R, and which may then modulate 5HT₂R signaling. A cell-based system may be used for such screening utilizing a CRFR1 or 5HT₂R-expressing cell line. The candidate compound is incubated with a CRFR1 or 5HT₂R-expressing cell line in the presence of SAP97, either co-expressed or otherwise present in the cells. The effect of the candidate compound on the interaction of SAP97 with CRFR1 or 5HT₂R is determined and compared to a control in which a CRFR1 or 5HT₂R-expressing cell line is incubated in the presence of SAP97 but in the absence of the candidate compound. For example, the effect on activation of ERK1/2 signaling by the CRFR1, binding of SAP97 to the PDZ binding motif of CRFR1 or 5HT₂R or receptor endocytosis may be determined using techniques well-established in the art such as those described in the specific examples that follow. A determination of an effect of the candidate compound on the SAP97 interaction with CRFR1/5HT₂R that differs from the control (e.g. normal) indicates that the candidate compound may be a potential therapeutic for use to desensitize 5-HT₂R signaling.

Embodiments of the invention are described in the following specific example which is not to be construed as limiting.

### Example 1

### De-Sensitization of 5-HT₂R Signaling

### Methods

Plasmid Constructs.

The FLAG-tagged human (h) 5-HT_{2C}R plasmid construct was generated by PCR and subcloned into pcDNA3.1 and the FLAG-tagged human (h) 5-HT_{2A}R plasmid construct was previously described, (Abbas et al. J. Neurosci. 29, 7124-7136 (2009)). The FLAG-5-HT_{2C}R-ASSV and HA-CRFR1-ΔTAV mutant receptors were constructed using the using the QuikChange™ site-directed mutagenesis kit (Stratagene). The HA-CRFR1 and GFP-Rab constructs were described previously (Holmes et al. J. Neurochem. 96, 934-949 (2006)). The CRFR2 cDNA clone was the kind gift of Dr. Wylie Vale.

Cell Cuture and Transfection.

HEK 293 cells were maintained in Eagle's minimal essential medium supplemented with 10% fetal bovine serum and gentamicin (100 μg/ml). Cells were seeded on 100 mm dishes at 80-90% density one day before transfection. Transfection was carried out using a modified calcium phosphate method as described previously (Conn et al. J. Pharmacol. Exp. Ther. 234, 195-203 (1985)). After transfection (approximately 17 hours), cells were washed with phosphate buffered saline (PBS), pooled and reseeded on appropriate dishes. Primary prefrontal cortical neurons were prepared from E18 CD1 mouse embryos as described previously (Holmes et al. 2006). Rat cortical neurons (R-cx-500, QBM cell science, Ottawa, Canada) were thawed and cultured for 6 days as suggested by manufacturers, then transfected with 4 μg of plasmid DNA encoding each receptor using lipofectamine. The University of Western Ontario Animal Care Committee approved all animal protocols.

Inositol Phosphate Formation.

Inositol phosphate formation in HEK 293 cells and mouse cortical neurons was determined by labeling cellular inositol lipids with 1 μCi/ml [³H] myo-inositol as previously described (Dhami et al. J. Biol. Chem. 279, 16614-16620 (2004)). Cells were then preincubated in either the presence or absence of CRF peptide for 30 min at 37° C. and then stimulated with increasing concentrations (0-10 μM) of 5-HT for 30 min at 37° C. Total [³H] inositol phosphate was purified from cell extracts by anion exchange chromatography [³H] inositol phosphate formation was determined by liquid scintillation counting as previously described (Dhamie et al. ibid). For inositol phosphate formation assay in brain slices, the protocol described by Conn and Sanders-Bush, (ibid) was utilized with minor modifications. Briefly, prefrontal cortex was isolated and cross-chopped (350×350 μm). Slices were suspended in Krebs Ringer Buffer (KRB) (108 mM NaCl 4.7 mM KCl, 1.2 mM MgSO₄, 1.2 mM KH₂PO₄, 2.5 mM CaCl₂, 25 mM NaHCO₃, 10 mM Glucose) and incubated for 30 min at 37° C. in a shaking bath under an atmosphere of O₂/CO₂ (95:5). Slices were then washed 3 times with 15 ml warm KRB and incubated with 5 μCi/ml [³H] myo-Inositol for 90 min (200 μl of gravity packed slices per ml of KRB). To remove excess radioactive inositol, slices were washed with 40 volumes of warm KRB containing 10 mM unlabeled myo-Inositol and allowed to settle under gravity. Buffer was aspirated off and 30 μl of gravity packed slices were aliquoted into tubes containing 240 μl of KRB containing 10 mM LiCl, 10 μM pargyline and ascorbic acid (100 μM). Slices were incubated for 15 min at 37° C. Following LiCl incubation, slices were preincubated in the presence or absence of 500 nM CRF peptide for 45 min at 37° C. (final volume=270 μl). Slices were then stimulated with 5-HT for 45 min (final volume=300 μl). The reaction was terminated by the addition of 3 volumes of chloroform/methanol (2:1, v/v) for 15 min at room temperature. One volume each of chloroform and HCl 0.15N was then added and the tubes were vortexed for 1 min. The phases were separated either by centrifugation at 1600 rpm for 5 min. Total inositol phosphate was purified from slice extracts by anion exchange chromatography as described above. Raw data was normalized for protein content which was measured in triplicate samples of prelabeled slices using the Bio-Rad D_{c} Protein Assay Kit following the manufacturer's instructions.

cAMP Assay.

Protocol was carried out as suggested by manufacturer. Briefly, HEK 293 cells transiently expressing FLAG-5-HT_{2A}R and HA-CRFR1 were seeded into 96-well plate (10,000 cells/well). Two days after transfection, cells were incubated in the absence or presence of 10 μM 5-HT in induction buffer [HBSS, 500 μM isobutyl-1-methylxanthine (IBMX)] for 30 minutes at 37° C. Cells were then incubated with increasing concentrations of CRF peptide for 30 minutes. Following stimulation, cells were solubilized with cAMP-Glo lysis buffer for 15 minutes with gentle shaking at room temperature. Lysates were carefully transferred to a white opaque 96-well plate and cAMP-Glo Detection solution containing protein kinase A was added for 20 minutes at room temperature followed by addition of Kinase-Glo Reagent for 10 min. Luminescence was measured using a Victor Reader (Perkin-Elmer, Walthan, Mass.).

Immunofluorescence Microscopy.

Immunofluoroescence was done as previously detailed on wt or 5-HT2A KO mice (Abbas et al. ibid). In brief, mice were transcardialy perfused with 4% paraformaldehyde in 1×PBS. Brains were then harvested and placed 12 h in 4% paraformaldehyde in 1×PBS at 4° C. and then placed in 30% sucrose in 1×PBS until they sank, then frozen on dry ice and stored at −80° C. Sections (30 μm) were free-floating in 1×PBS (one per well in a 24-well plate) and then permeabilized with 0.4% Triton X100 in 1×PBS for 1 hour. PBS 1×/0.4% Triton X100 containing 0.1% glycine, 0.1% lysine, 1% BSA and 1% normal donkey serum. Primary antibodies (anti 5-HT_{2A}, rabbit polyclonal, Neuromics cat #RA24288 and anti CRFR1, goat polyclonal, Abcam cat #ab59023) were incubated in blocking buffer for 72 hours at 4° C. Sections were then washed five times in 1×PBS/0.4% triton (10 min each). Hoechst (2-5 μg/ml) and secondary antibodies: donkey Alexa Fluor 555 conjugated anti-goat (1:500) and donkey Alexa Fluor 488 conjugated anti-rabbit antibodies (1:500) (Invitrogen) were diluted in blocking buffer and slices were incubated for 1 hour at RT. Sections were washed five times in 1×PBS/0.4% triton (10 min each). Sections were mounted on slides and visualized by Zeiss LSM-510 META multophoton laser scanning microscope with a Zeiss 25×NA 1.2 oil immersion lens and appropriate filters.

Biotinylation of Cell Surface Receptor.

HEK 293 cells transiently expressing wild-type and truncated FLAG-5-HT_{2A}R and HA-CRFR1 were seeded into 100 mm dishes and pre-incubated for 30 minutes in HBSS. Cells were then treated for 30 min with or without 500 nM CRF, washed twice with ice-cold HBSS and placed on ice for biotin labeling. Cell surface receptors were labeled on ice with biotin (1 mg/ml) for 1 hour. Following labeling, cells were washed 3 times with 10 mM glycine and then 2 times with HBSS, lysed and equal amounts of total protein were incubated with neutravidin beads for 2 hours with rotation at 4° C. Beads were then washed 3 times with lysis buffer and one time with PBS. Proteins were eluted from beads by addition of 50 ul of SDS loading buffer. Samples were resolved by SDS-PAGE, transferred to nitrocellulose membrane and subsequently immunoblotted as described above with rabbit polyclonal FLAG antibody.

Surgical Procedure.

Male CD-1 mice were obtained from Charles River Canada (St. Constant, Quebec) at 50-60 days of age, and were acclimatized to the laboratory for approximately 30 days before serving as experimental subjects. Mice were housed four per cage, until the time of surgery, after which they were housed individually. The vivarium was maintained on a 12-h light/dark cycle in a temperature (21° C.) controlled room with food and water freely available. Mice were anesthetized using isoflurane and stereotaxic surgery (David Kopf Instruments Model 940) was performed to install a cannulae into the medial prefrontal cortex. A Guide cannulae (Plastics One In), situated according to the mouse atlas of Franklin and Paxinos at Lateral=0.32 mm, D/V=2.25 mm, A/P=+2.68 mm. A dummy cannula, was inserted flush with guide. Approximately 1 week after behavioral testing mice were perfused with 4% paraformaldehyde. Brains were subsequently sectioned at 14 microns and stained with Cresyl violet for probe placement verification. Only the data from mice with correct probe placements were used in the analysis of the behavioral tests.

Drug Treatments.

One week after surgical recovery animals were infused with 1.0 μl of CRF (1.5 μg) or vehicle (Phoenix Pharmaceuticals) over a 5 min period through an internal cannulae situated 0.3 mm below the guide cannulae. Drug diffusion was permitted for 5 min, and then after a further 5 min period mice were injected intraperotineally with DOI (Sigma) at a dose of 0.15 mg/kg or saline. Behavioral testing was conducted 15 min after the DOI treatment. In a second experiment the procedure was identical to that of the preceding study, except that mice were pretreated i.p. with either vehicle or 0.25 mg/kg of M100907 in a volume of 0.3 ml immediately prior to the DOI treatment. As in the preceding study mice were then tested in the elevated plus maze test (n=6-8)/group. Once again, data were obtained from videotapes and the researcher was blind as to the treatments mice had received.

Behavioral Testing.

In an initial test, mice were placed in a 45×45 cm open field, with an inner square of 21×24×24 cm., for a 5 min period, during which the time to enter the center area, and the total time spent in the center portion of the arena was recorded. The plus maze test was then conducted 1 min after the open field assessment. Mice were individually placed in one of the enclosed arms of a plus-maze and the behavior of the animals was recorded over a 5 min period by a ceiling-mounted video camera. The amount of time spent in each of the arms, the number of arm entries (an arm entry was defined as all four of the paws being placed in an arm of the plus-maze). The elevated plus-maze had two arms enclosed by 21 cm high walls; whereas the remaining two arms were open (arms were 24.8 cm long×7.7 cm wide). The maze was situated in a dimly lit room, such that the closed arms were darkened, whereas open arms were somewhat illuminated. All behavioural experiments were blinded. All experiments complied with the guidelines set by the Canadian Council on Animal Care and were approved by the Carleton University Animal Care Committee.

Data Analysis.

The mean and the standard error of the mean were expressed for values obtained from the number of separate experiments indicated. Dose response data were analyzed using GraphPad Prism (GraphPad Software). Statistical significance was determined by analysis of variance and corrected for multinositol phosphatele comparisons. For behavioural testing data were analyzed by either a two factor (Drug infusion and DOI treatment) or three factor (Drug indusion, DOI treatment and M100907) analysis of variance (ANOVA), as appropriate, independently for each of the outcome measures. Follow-up tests were conducted by Bonferonni t tests corrected to maintain the a at 0.05.

### Results

CRFR1 Activation Enhances 5-HT₂R Signaling.

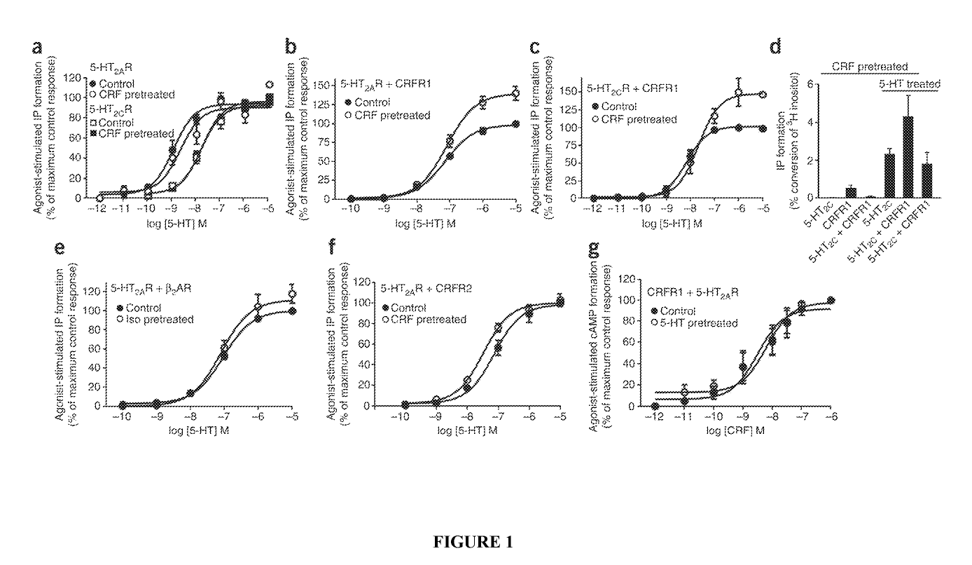

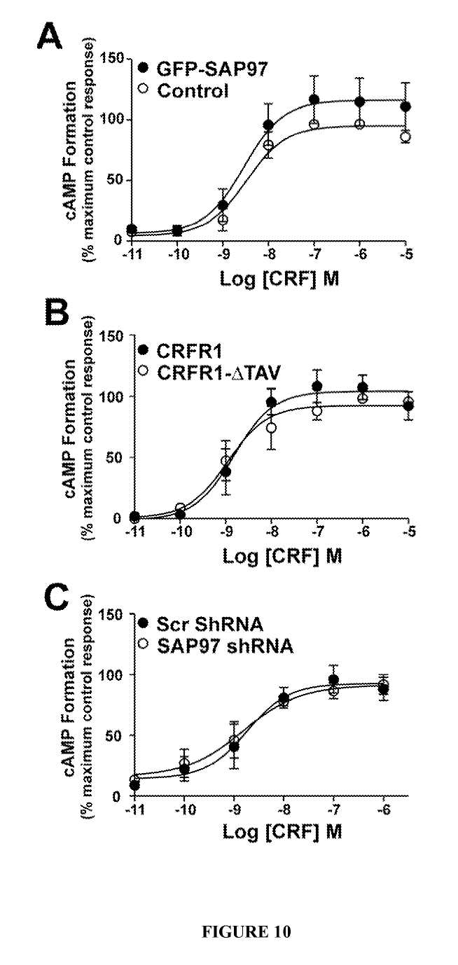

The mechanism by which CRFR1, a receptor coupled Gαₛ-stimulated cAMP accumulation, might alter the signaling of Gα_{q/11}-coupled receptors (5-HT_{2A}R and 5-HT_{2C}R) that stimulate increases in inositol phosphate formation was studied. Human embryonic kidney (HEK 293) cells that do not express endogenous CRFR1 or 5-HT₂Rs were used to examine whether CRFR1 activation altered 5-HT₂R signaling. In HEK 293 cells transfected to express either 5-HT_{2A}R or 5-HT_{2C}R in the absence of CRFR1, the treatment of cells with increasing concentrations of 5-HT resulted in a dose-dependent increase in inositol phosphate formation and pretreatment with CRF had no effect on the dose-response curves for inositol phosphate formation for either receptor (FIG. 1a). However, in cells expressing either 5-HT_{2A}R or 5-HT_{2C}R along with CRFR1, CRF pretreatment (500 nM) for 30 min increased the maximum efficacy (E_{MAX}) for both 5-HT_{2A}R- and 5-HT_{2C}R-stimulated inositol phosphate formation by 40±4.7% and 47±5.5%, respectively (FIG. 1b,c). The increase in 5-HT₂R-mediated inositol phosphate formation observed following CRF pretreatment was not attributable to CRFR1-mediated inositol phosphate formation, as CRF treatment of HEK 293 cells for 30 min did not result in inositol phosphate formation in cells expressing the 5-HT_{2C}R alone, CRFR1 alone or expressing both receptors (FIG. 1d). To determine whether the observed enhancement in 5-HT₂R signaling was specific to CRFR1, it was determined whether the coexpression and activation of another Gαₛ-coupled GPCR also increased 5-HT₂R signaling. However, in HEK 293 cells expressing both the β₂-adrenergic receptor (β₂AR) and 5-HT_{2A}R, isoproterenol (100 μM) pretreatment had no effect on the magnitude of 5-HT_{2A}R-stimulated inositol phosphate responses (FIG. 1e). Similarly, in cells co-expressing CRFR2 and 5-HT_{2A}R, CRF pretreatment did not increase 5-HT_{2A}R-stimulated inositol phosphate responses (FIG. 10. When the activation of the 5-HT_{2A}R was examined to determine if it might increase CRFR1-mediated cAMP formation, it was found that 5-HT (10 μM) pretreatment had no effect on CRFR1 responsiveness (FIG. 1g). In addition, the effect of inhibiting either cAMP-dependent protein kinase (PKA) or protein kinase C (PKC) that are activated by CRFR1 and 5-HT₂R, respectively, was examined and it was found that inhibition of either kinase had no effect on CRFR1-mediated increases in 5-HT_{2C}R signaling. Thus, CRFR1 activation lead to increased 5-HT₂R signaling and this increased 5-HT₂R signaling was unique to CRFR1 and could not be mimicked by another Gαₛ-coupled GPCR.

It was essential to establish whether the augmented 5-HT₂R signaling in response to CRF was observed in prefrontal cortical neurons. Therefore, it was determined whether or not both receptors were expressed in neurons from the prefrontal cortex of mice. Mouse prefrontal cortical slices were stained with polyclonal antibodies that recognized either endogenous 5-HT_{2A}R or CRFR1 and Hoechst to mark cell nuclei. It was found that a subpopulation of neurons in the prefrontal cortex stained positive for both 5-HT_{2A}R and CRFR1 protein. The specificity of the 5-HT_{2A}R antibody was confirmed in parallel Western blot and immunofluorescent studies of prefrontal cortex from 5-HT_{2A}R knockout mice. CRFR1 antibody specificity was confirmed in HEK293 cells expressing HA-CRFR1. CRF (500 nM) pretreatment of mouse neuronal cultures for 30 min was found to significantly increase 5-HT (50 μM)-stimulated [³H]-myo-inositol conversion to inositol phosphate. Importantly, in slices prepared from prefrontal cortex CRF pretreatment increased 5-HT-stimulated inositol phosphate formation by 2.3±0.2 fold and when the 5-HT_{2A/C}R selective agonist 2,5-dimethoxy-4-iodoamphetamine (DOI; 10 μM) was used, CRF pretreatment increased inositol phosphate formation by 1.5±0.2 fold. Thus, consistent with what was observed in an overexpression system the pretreatment of endogenous CRF receptor increased 5-HT/DOI-stimulated inositol phosphate formation in prefrontal neuronal cultures and tissue.

### Mechanism Underlying CRF-Mediated Increases in 5-HT₂R Signaling

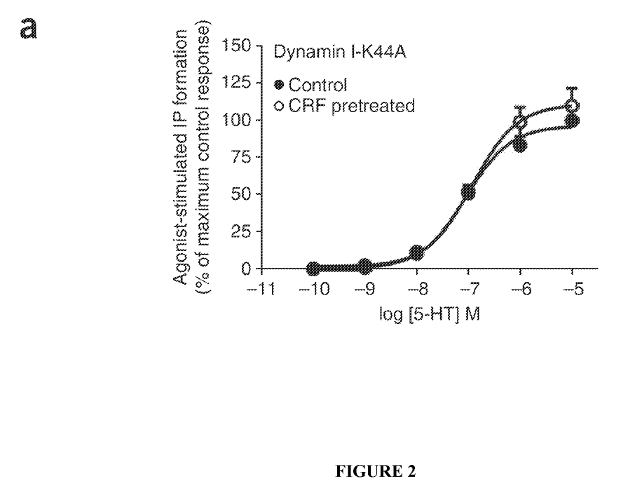

The sensitization in 5-HT₂R signaling was unique to CRFR1 and was independent of the activity of second messenger-dependent protein kinases activated by either receptor. Therefore, it was examined whether or not agonist-stimulated CRFR1 internalization contributed to the sensitization of 5-HT₂R signaling. It was first determined whether the expression of a dominant-negative inhibitor of clathrin-mediated endocytosis (dynamin I-K44A) altered CRFR1-mediated increases in 5-HT_{2A}R signaling in HEK 293 cells. Dynamin I-K44A expression completely eliminated CRFR1-dependent increases in 5-HT_{2A}R-stimulated inositol phosphate formation following CRF pretreatment (FIG. 2a).

HA-epitope tagged CRFR1 and FLAG-epitope tagged 5-HT₂R that were immunofluorescently labeled at the cell surface at 4° C. were localized and then allowed to warm to 37° C. in both HEK 293 cells and rat cortical neurons. Both FLAG-5-HT_{2A}R and FLAG-5-HT_{2C}R were internalized from the cell surface in the absence of agonist, whereas no constitutive endocytosis was observed for the HA-CRFR1. Similarly, in transfected neurons FLAG-5-HT_{2A}R, but not CRFR1 was observed to internalize from the cell surface in the absence of agonist treatment. In contrast, when rat cortical neurons were warmed to 37° C. and treated with 100 nM CRF both HA-CRFR1 and FLAG-5-HT_{2A}R (untreated) were endocytosed and were colocalized within the same intracellular vesicles. Similar to what was observed for the HA-CRFR1, agonist-stimulated HA-β₂AR also colocalized with FLAG-5-HT_{2A}R in vesicles after isoproterenol treatment, but this does not translate into an alteration in 5-HT_{2A}R signaling (FIG. 1e). HA-CRFR1 and FLAG-5-HT_{2A}R were found to be colocalized to both Rab5- and Rab4-positive endocytic organelles. Thus, not only was the localization of the 5-HT₂R between the cell surface and intracellular compartments of cell dynamically regulated, CRFR1 endocytosis was required for the sensitization of 5-HT₂R responses to agonist.

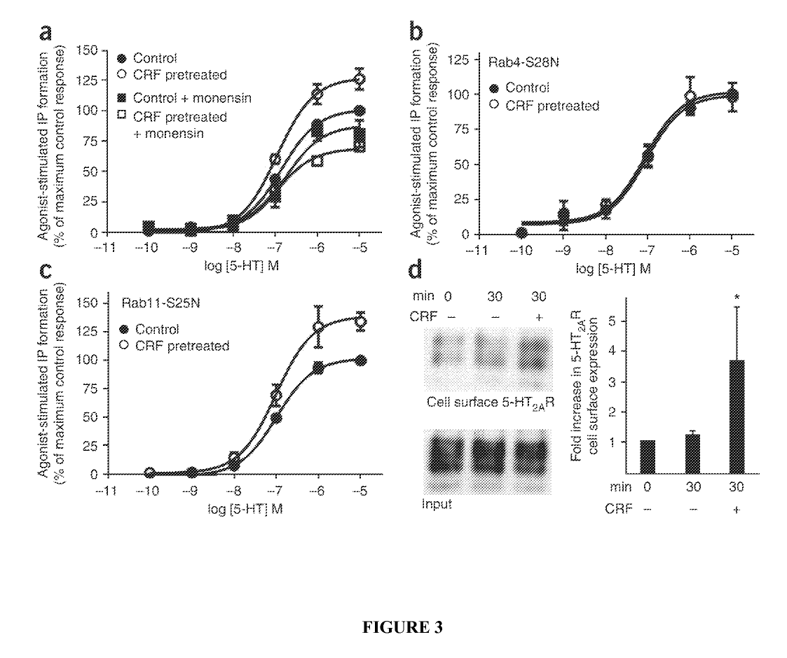

To further assess the role of the intracellular trafficking of both the 5-HT_{2A}R and CRFR1 in the CRF-dependent regulation of 5-HT_{2A}R signaling, inhibition of receptor recycling with monensin was tested to determined if it would block CRF-mediated increases in 5-HT_{2A}R signaling. Treatment of cells with 100 μM monensin did not affect 5-HT_{2A}R signaling in the absence of CRF pretreatment (FIG. 3a). However, monensin treatment attenuated the increase in 5-HT_{2A}R signaling observed following CRF pretreatment (FIG. 3a). To assess whether the effect of monensin treatment was related to the recycling of receptors through endosomes, dominant negative Rab4-S28N and Rab11-S25N proteins were utilized to selectively inhibit receptor recycling via rapid (Rab4 positive) and slow (Rab11 positive) recycling endosomes. Overexpression of Rab4-S28N, but not the overexpression of Rab11-S25N, blocked the increase in 5-HT_{2A}R-mediated inositol phosphate formation induced by CRFR1 pre-activation (FIG. 3b,c). Biotinylation of cell surface FLAG-5-HT_{2A}R also revealed that CRF pretreatment increased the cell surface expression of the 5-HT_{2A}R by 3.7±1.8 fold (FIG. 3d). Accordingly, the endocytosis and recycling of CRFR1 was essential for regulating 5-HT_{2A}R signaling via a mechanism that resulted in increased 5-HT_{2A}R expression at the cell surface.

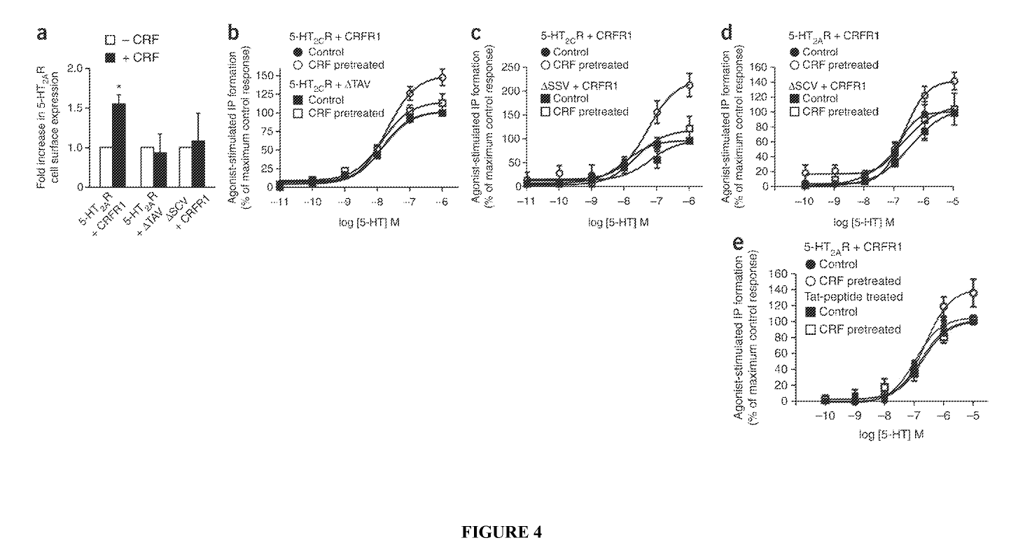

All three receptors encoded class I PDZ domain interacting motifs at the end of their carboxyl-terminal tails and both the 5-HT_{2A}R and 5-HT_{2C}R. Therefore, it was examined whether the deletion of three amino acids from the 5-HT_{2A}R (ΔSCV) and CRFR1 (ΔTAV) carboxyl-terminal tails would affect cell surface recruitment of the 5-HT_{2A}R following CRF treatment. When tested, the deletion of either the 5-HT_{2A}R or CRFR1 PDZ domain binding motifs attenuated the CRF-dependent increases in 5-HT_{2A}R at the cell surface (FIG. 4a). Since a loss of the PDZ binding motifs on either the 5-HT_{2A}R or CRFR1 resulted in a loss of CRFR1-dependent recruitment of 5-HT_{2A}R to the cell surface, it was tested whether PDZ domain interactions were required for CRFR1-mediated sensitization of 5-HT₂R signaling. Truncation of the final three amino acid residues of the CRFR1 carboxyl terminal tail (ΔTAV) prevented CRFR1-mediated increases in 5-HT_{2C}R signaling following CRF pretreatment (FIG. 4b). Similarly, increased 5-HT_{2c}R inositol phosphate formation in response to CRFR1 activation was not observed following the deletion of either the 5-HT_{2C}R PDZ (ΔSSV) or 5-HT_{2A}R (ΔSCV) domain binding motifs (FIG. 4c,d). The treatment of HEK293 cells with a peptide that encoded the HIV Tat protein membrane transducing domain fused to the last 10 amino acid residues corresponding to the CRFR1 carboxyl-terminal tail (NOTE: so the 10-aa CRFR1 carboxy tail peptide bound to the 5-HT2R tail to prevent sensitization of 5-HT2R? Pls confirm) prevented CRFR1-mediated sensitization of 5-HT_{2A}R signaling (FIG. 4e). Thus, intact PDZ domain protein interactions with both receptors were required for CRFR1-dependent sensitization of 5-HT₂R responses.

### CRF Treatment Enhances 5-HT-Mediated Anxiety-Related Behaviours

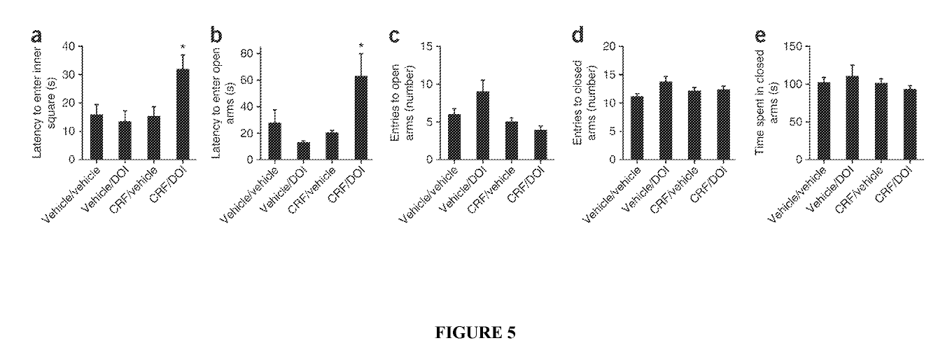

To assess the role of CRF in the regulation of 5-HT₂R-mediated anxiety behaviour, two anxiety-related behaviours were examined in mice: (1) the latency for mice to enter the center of an open field and (2) the latency for mice to enter the open arm of an elevated plus maze. Having established the molecular mechanism by which CRFR1 activation sensitized 5-HT₂R responses in vitro, it was determined whether or not the infusion of CRF peptide (1.5 μg) into the medial prefrontal cortex followed by the intraperitoneal administration of the 5-HT₂R selective agonist DOI (0.15 mg/kg) would sensitize 5-HT-mediated anxiety-related behavioral responses. The latency of mice to enter the center of an open field varied as a function of the intracerebral infusion (CRF vs vehicle) x systemic challenge (DOI vs vehicle) interaction, F(1,35)=7.01, p<0.01. Follow-up analysis of the mean latencies for mice to enter the center square in a 5 min open field test revealed that neither the CRF nor the DOI treatments alone influenced performance relative to the vehicle-vehicle condition (FIG. 5a). However, among mice that received both CRF and DOI treatment the latency to enter the central portion of the maze was significantly longer than that of mice that received only a single drug treatment or vehicle (FIG. 5a). In the plus-maze test, the latency to enter an open arm, as well as the number of entries onto the open arms, also varied as a function of the IC infusion (CRF vs vehicle) x systemic challenge (DOI vs vehicle) interaction, F(1,35)=7.85, 3.89, p<0.01 and 0.05, respectively. Follow-up comparisons indicated that DOI itself produced a modest reduction in the latency to enter an open arm (p<0.08) and the number of arm entries emitted (p<0.10), whereas CRF infusion had no effect (FIG. 5b,c). However, among mice that received both the CRF and DOI treatments a marked increase of the open arm latency and a decreased frequency of open arm entries was evident relative to mice that received either treatment alone (FIG. 5b,c). In contrast to these findings, the number of entries into the closed arms, which approximately doubled the open arm entries, did not vary with either the CRF or DOI treatments, or as a function of their interaction (p>0.15) (FIG. 5d). Likewise, the time spent in the closed arms did not vary as a function of the treatments mice received (F<1) (FIG. 5e).

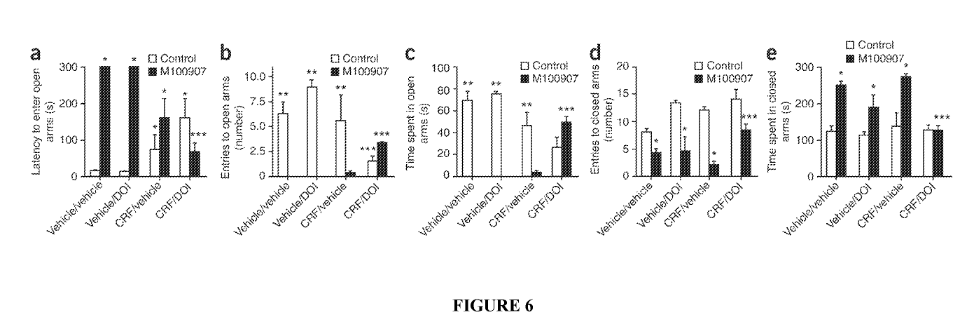

In a follow up series of experiments it was determined whether or not the synergistic effects of DOI and CRF treatment could be antagonized by the pretreatment of mice with the 5-HT_{2A}R selective antagonist M100907. The latencies to enter the open arms of the plus maze varied as a function of the DOI×CRF×M100907 interaction, F(1,41)=6.00, p=0.018 (FIG. 6a). The tests confirmed that treatment with DOI alone did not influence the latencies to enter the open arms, whereas CRF infusion provoked a moderate, but statistically significant increase in response latencies. In mice that received the combination of systemic DOI following CRF administration to the prefrontal cortex, latencies to enter the open arms were still longer (FIG. 6a). When mice were treated with M100907 alone or with M100907 plus DOI none of the mice entered the open arms of the plus maze. Likewise, when given M100907 in conjunction with CRF, latencies were longer than in mice that received CRF alone, although several mice did enter onto the open arms (FIG. 6a). As predicted, when mice received M100907 in conjunction with DOI and CRF the latencies to enter the open arms of the maze were markedly reduced from that elicited by the combination of DOI plus CRF. Thus despite the fact that M100907-treated mice displayed a significant reluctance to enter the open arms of the maze, M100907 effectively attenuated the effects of the DOI-CRF combination.

The analysis of both the number of open-arm entries and the time spent in the open arms revealed responses which paralleled that of the response latencies (FIG. 6b,c). Specifically, the DOI×CRF×M100907 interaction was highly significant, F(1,41)=10.78, 15.04, p<0.001, and the follow up tests confirmed that neither CRF nor DOI alone affected the frequency of open arm entries. By contrast the combination of these treatments significantly reduced open arm entries and reduced the time spent in the open arms, as observed in the preceding studies. The M100907 profoundly influenced the frequency of open arm entries and time spent on the open arms (as described in the analysis of the latencies) in that mice treated with the compound (alone or in combination with DOI) did not make any entries onto the open arm, and most animals treated with M100907 and CRF also failed to make open arm entries (FIG. 6b,c). However, when animals received all three compounds, open arm entries and time on the open arms increased significantly relative to mice that either received DOI and CRF (but not M100907) or those that received CRF and M100907 (but not DOI). However, the number of entries were clearly fewer than that of animals that were either untreated or that had received only DOI (FIG. 6b).

The analysis of the entries to the closed arms indicated that behavior was significantly influenced by the DOI×CRF×M100907 interaction, F(1,41)=9.29, p<0.01 (FIG. 6d). The follow up tests indicated that DOI, CRF and the combination of these treatments increased closed arm entries relative to mice that had received only the vehicle treatments. Thus, one cannot ascribe the reduced open arm entries induced by the CRF-DOI combination to reduced motor activity. The M100907 treatment alone reduced the frequency of arm entries, irrespective of the other treatments received, although the magnitude of this effect was less pronounced in mice that had also received DOI+CRF. The time spent in the closed arms was unaffected by either the DOI or CRF or their combination (FIG. 6e). However, time spent in the closed arms was increased by M100907 in those mice that received this treatment alone, or either DOI or CRF. However, time spent in the closed arms among mice that received the combination of the three treatments did not differ from that of mice that received the CRF+DOI or those that received DOI+M100907. However, the time spent in the closed arms among mice that received the combination of DOI, CRF and M100907 was indistinguishable from that of mice that received only vehicle, or either CRF or DOI alone (FIG. 6e). Taken together this data in mice showed that CRFR activation resulted in increased 5-HT₂R signaling in vivo and that the activation of both receptors had an important effect on behavioural responses associated with anxiety.

### Discussion

CRF was shown to act through CRFR1 to sensitize 5-HT₂R-mediated signaling and anxiety behaviours thereby linking CRF-mediated stress responses to anxiety and depression. The present findings indicate that enhanced 5-HT₂R sensitivity following CRF pretreatment in vivo was evidenced by increased anxiety-related behaviour in mice. This observation showed that CRF could potentiate 5HT₂R mediated behaviours and has implications regarding the mechanisms by which stressors may exacerbate the anxiogenic effects of 5HT₂R activation. Importantly, the behavioural data, which showed a functional interaction between CRF and 5-HT, were supported at the cellular level. Thus, it was demonstrated both that CRFR1 activation positively modulated 5-HT₂R signaling in cortical neurons and that these two receptors were co-expressed in the same neuronal populations. The molecular mechanism underlying the sensitization of 5-HT₂R signaling by CRFR1 required agonist-stimulated CRFR1 endocytosis and recycling which resulted in increased cell surface expression of 5-HT₂Rs and increased second messenger responses to 5-HT treatment. These findings provide an additional mechanism by which receptor endocytosis and recycling contribute to the regulation of GPCR responsiveness in general and specifically show how CRFR1 activation can positively modulate 5-HT₂R signaling thereby leading to pathophysiological behavioural responses.

Anxiety responses in both an open field emergence and in a plus-maze test were shown to be sensitized in mice that were pretreated with CRF administered to the prefrontal cortex, followed by systemic administration of a low dose of DOI. When administered alone, neither of these treatments affected performance in these tests, demonstrating that the CRF and DOI treatments acted synergistically to provoke the anxiety responses. The behavioral change could not be attributed to diminished motoric activity, as entries into the closed arms of the plus-maze were unaffected by the treatments. It should be said that when significantly higher doses of DOI were employed (0.625 and 1.25; data not shown) elevated arm entries were evident (as opposed to reduced open-arm entries), likely reflecting an overall arousal.

Thus, both the CRF and 5-HT systems when sufficiently activated will independently lead to anxiety responses. The 5-HT_{2A}R selective antagonist M100907 itself also provoked marked reductions of open arm entries suggesting that M100907 could independently induce an anxiety-like response. As entries into the closed arm were observed, it was clear that the absolute failure to enter the open arm was not due to motor impairments, and instead it was likely that the reduced activity reflected an overall increase of anxiety. However, of particular significance, was the observation that the anxiety-provoking effects of CRF and DOI cotreatment were antagonized by M100907 pretreatment. Thus, the observations indicated that cross-talk between CRF- and 5-HT-mediated signaling processes occurred in the prefrontal cortex and that CRF sensitized 5-HT₂-processes to promote stressor-like effects, such as anxiety.

Based on the present data, a multistep mechanism is proposed whereby CRF peptide activation of CRFR1 enhances 5-HT₂R signaling by increasing the availability of 5-HT₂R at the surface of cells to be activated by agonist and to couple to the activation of phospholinositol phosphatase Cβ-mediated inositol phosphate formation. Agonist-activation of CRFR1 promoted the dynamin-dependent internalization of CRFR1 into the intracellular endosomal compartment of the cell and 5-HT_{2A}R and 5-HT_{2C}R were internalized to endosomes in a constitutive manner. Thus, following agonist treatment internalized CRFR1 facilitated the cell surface recycling of 5-HT₂R from endosomes resulting in increased 5-HT₂R protein at the cell surface. The CRFR-dependent enhancement of 5-HT₂R signaling also required the interaction of PDZ domain containing proteins with both receptors, since the deletion of PDZ binding motifs in the carboxyl-terminal tail domains of either CRFR1, 5-HT_{2A}R or 5-HT_{2C}R prevented CRF-mediated sensitization of 5-HT₂R signaling. Interestingly, the activation of CRFR2, another CRFR expressed in the brain, did not sensitize 5-HT_{2A}R signaling and consistent with this observation examination of the CRFR2 carboxyl-terminal tail revealed that the canonical PDZ binding motif was disrupted.

Sensitization of 5-HT₂R signaling was dependent on receptor endocytosis as dynamin I-K44A expression could block this effect. This suggested that the internalization of either the CRFR1 or the 5-HT₂Rs was essential for sensitizing 5-HT₂R signaling. Several lines of evidence suggest that it is the internalization of CRFR1 that is essential for this effect. First, both 5-HT_{2A}R and 5-HT_{2C}R are found to be predominantly intracellular in neurons of the rat prefrontal cortex. Second, in the present study, it was found that both 5-HT_{2A}R and 5-HT_{2C}R were constitutively internalized in both HEK 293 cells and neurons, although cell surface expression of 5-HT_{2A}R has been reported. However, the mechanism underlying the observed constitutive endocytosis was unclear and may be a consequence of the fact that the serum used to culture cells may contain 5-HT. Independent of the mechanism by which 5-HT₂R were internalized, it is proposed that it was the internalization and recycling of the CRFR1 that dynamically regulated the subcellular equilibrium of 5-HT₂R resulting in the redistribution of 5-HT₂R to the cell surface resulting in the sensitization of 5-HT₂R signaling.

The CRFR1-mediated increases in 5-HT_{2A}R signaling were also blocked by either the treatment of cells with monensin, which prevents the trafficking of intracellular vesicles or the overexpression of a dominant-negative Rab4-S28N mutant protein that blocked rapid recycling of GPCRs to the cell surface. Thus, CRFR1 sensitization of 5-HT₂R signaling required increased 5-HT₂R recycling and cell surface expression. The intracellular localization of 5-HT₂R may prevent over-stimulation of serotonergic synapses. The regulated recruitment of this intracellular pool of 5-HT₂R may function to promote altered post-synaptic signal adaptation to physiological stimuli, such as CRF peptide release in response to stress leading to the activation of CRFR1 in 5-HT₂R expressing neurons of the prefrontal cortex.

CRFR1-dependent alterations in 5-HT₂R signaling required intact PDZ binding motifs at the carboxyl-terminal tails of both CRFR1 and 5-HT₂Rs. Examples of PDZ domain containing proteins that interact with both 5-HT_{2A}R and 5-HT_{2C}R include MAGI-2, MPP3, MUPP1, PSD-95 and SAP97. Each of these PDZ domain containing proteins are comprised of multiple PDZ domains that would allow them to form complexes with more than one GPCR.

### Example 2

### Inhibition of PDZ Domain-Containing Protein, SAP97

### Experimental Procedures

Materials:

Goat anti-glutathione-S-transferase (GST) antibodies as well as ECL Western blotting detection reagents were purchased from GE Healthcare (Oakville, ON, Canada). Rabbit anti-phospho-p44/42 MAP kinase (Thr202/Tyr402), and rabbit anti-p44/42 MAP kinase antibodies were obtained from Cell Signalling Technology (Pickering, ON, Canada). Rabbit anti-GFP antibody was obtained from Invitrogen/Life Technologies (Burlington, ON, Canada). Mouse anti-SAP97 antibody was obtained from Assay Designs/Enzo Life Sciences (Farmingdale, N.Y., USA). Alexa Fluor 647 anti-mouse IgG and Alexa Fluor 633 goat anti-mouse IgG Zenon antibodies were purchased from Invitrogen/Molecular Probes (Burlington, ON, Canada). cAMP GLO Assay was obtained from Promega (Madison, Wis., USA). Mouse anti-HA antibody and all other biochemical reagents were purchased from Sigma-Aldrich (Oakville, ON, Canada).

Plasmid Constructs:

The HA-CRFR1 and HA-CRFR1ΔTAV receptors were described previously (Holmes et al. (2006) J. Neurochem. 96, 934-949). The YFP-SAP97 and SAP97 single hairpin RNA (shRNA) constructs were graciously provided by Dr. Suleiman W. Bahouth (Neuroscience Institute, University of Tennessee Health Sciences Center) (Gardner et al. (2007) J. Biol. Chem. 282, 5085-5099.). The YFP-SAP97 was subcloned into the pEGFP1 vector. The EPAC cAMP biosensor was the gift of Drs. Ali Salahpour (University of Toronto) and Marc Caron (Duke University) (Barak et al. (2008) Mol. Pharmacol. 74, 585-594). The CRFR1 carboxyl-terminal tail was cloned into pGEX-4 with ECOR1/NotI.

Cell Culture and Transfection:

Human embryonic kidney (HEK 293) cells were maintained in Eagle's minimal essential medium supplemented with 10% fetal bovine serum. Cells were seeded on 10 cm dishes at 70-80% density 24 h prior to transfection. Transfection was performed using a modified calcium phosphate method, as described previously (Ferguson et al. (2004) Methods Mol. Biol. 237, 121-126). 1 μg of each construct was used for each transfection, with the exception that 3 μg of plasmid cDNA was used for all shRNA constructs. Empty pcDNA3.1 vector was used to equalize the total amount of plasmid cDNA used to transfect cells. 18 h post-transfection, cells were washed with phosphate buffered saline (PBS) and re-suspended with Trypsin 0.25% EDTA. Cells were then reseeded for experimentation. All experiments were conducted approximately 48 h after the initial transfection, with exception of transfections involving SAP97 shRNA, which were conducted 72 h after initial transfection to optimize the knockdown of endogenous SAP97, as confirmed by Western blotting.

PDZ Blot Overlay Assay:

GST and GST-CRFR1 peptides were generated by growing recombinant BL21 bacteria at 21° C. to an A₆₀₀ of 0.6-1.0. Cultures were induced for 3 hrs with 1 mM IPTG, pelleted, resuspended in PBS containing protease inhibitors (1 mM AEBSF, 10 g/ml leupeptin, and 5 μg/ml aprotinin) and lysed by mild sonication. The bacterial lysates were cleared of cellular debris by centrifugation and then applied to Glutathione Sepharose 4B overnight at 4° C. GST and GST-CRFR1 peptides bound to the matrix were washed extensively in PBS-containing 0.3% Triton X-100. 100 nM of GST and GST-CRFR1 peptide in blot buffer (2% nonfat dry milk, 0.1% Tween 20, 50 mM NaCl, 10 mM Hepes, pH 7.4) were incubated with gridded nylon membranes that were spotted with His/S-tagged PDZ domain fusion proteins (1 μg/bin) for 1 h at room temperature. The arrays were. The arrays were then washed three times with blot buffer, and incubated with a horseradish peroxidase-conjugated anti-GST antibody (1:3000). Interactions of the GST fusion proteins with the various PDZ domains were then visualized via chemiluminescence using the enhanced chemiluminescence kit from GE Healthcare.

Co-Immunoprecipitation:

Transfected HEK 293 cells were seed on 10 cm dishes the day before the experiment. Cells were serum-starved for 1 hour in HEPES-buffered saline solution (HBSS), and dishes were treated with either HBSS alone or with 100 nM CRF agonist in HBSS for 30 min at 37° C. Cells were subsequently lysed in lysis buffer (50 mM Tris, pH 8.0, 150 mM NaCl, and 0.1% Triton X-100) containing protease inhibitors (1 mM AEBSF, 10 μg/ml leupeptin, and 5 aprotinin) for 20 min on a rocking platform at 4° C. Samples were collected into 1.5 mL Eppendorf tubes and centrifuged at 15,000 g for 15 min at 4° C. to pellet insoluble material. A Bronsted-Lowry protein assay was performed and 400 μg of protein was incubated for 1-2 h at 4° C. with Protein G Sepharose and mouse anti-HA antibody (1:50). After incubation, beads were washed 3 times with cold lysis buffer and incubated overnight at room temperature in 3×SDS Loading Buffer containing 2-mercaptoethanol. Samples were separated by SDS-PAGE, transferred to a nitrocellulose membrane, and immunoblotted to identify co-immunoprecipitated GFP-SAP97 (rabbit anti-GFP, 1:1000). An additional Western blot was performed to examine HA-CRFR1, HA-CRFR1ΔTAV (mouse anti-HA, 1:1000) and GFP-SAP97 (rabbit anti-GFP, 1:1000) protein expression.

Live HEK293 Cell Immunofluorescent Confocal Microscopy:

Following transfection, HEK 293 cells were re-seeded on 35-mm glass-bottom confocal dishes. Cells were serum-starved for 1 hour at 37° C. in HBSS, then labeled with mouse anti-HA antibody (1:200) and Zenon Alexa Fluor 647 mouse IgG1 Labeling Kit (Invitrogen) at 4° C. for 30 minutes. The cells were washed with HBSS and warmed to 37° C. for live imaging using a heated stage. Confocal microscopy was performed on a Zeiss LSM-510 META laser scanning confocal microscope using a Zeiss 63×, 1.3 NA, oil immersion lens. Co-localization studies were performed using dual excitation (488 nm, 633 nm) and emission (band pass 505-550 nm and long pass 650 nm for YFP/GFP and Alexa Fluor 647, respectively) filter sets. The specificity of labeling and absence of signal crossover were established by examination of single-labeled samples. Receptor endocytosis experiments were additionally stimulated with 500 nM CRF agonist (Tocris) and specified cells were re-imaged at regular intervals for up to 60 minutes.

Receptor Endocytosis:

Following transfection, HEK 293 cells were re-seeded into 12-well plates. Cells were serum-starved for 1 h at 37° C. in HBSS and then stimulated for certain periods of time with or without 500 nM CRF in HBSS at 37° C. Cells were washed with cold HBSS and treated with mouse anti-HA antibody (1:500) for 45 min on ice. Cells were washed with cold HBSS and additionally treated with Alexa Fluor 633 goat anti-mouse IgG (Invitrogen) (1:500) for 45 min on ice. Cells were washed with cold PBS and treated with 5 mM EDTA in PBS for 5 min on ice. Newly suspended HEK 293 cells were then transferred to flow cytometry tubes containing 4% formaldehyde in PBS. Samples were run on a FACSCalibur cytometer using BD CellQuest Pro software until 10,000 cells were counted. The geometric mean of fluorescence was determined using FlowJo analysis software, with less fluorescence corresponding to less CRFR1 on the membrane.

cAMP Assay:

The cAMP GLO Assay protocol was carried out as suggested by the manufacturer (Promega). Transfected HEK 293 cells were seeded into 96-well plate (˜10,000 cells per well). Cells were incubated in induction buffer (HBSS with 500 μM isobutyl-1-methylxanthine (IBMX) and increasing concentrations of CRF agonist for 30 min at 37° C. Following stimulation, cells were solubilized with cAMP-GLO Lysis Buffer for 15 min with gentle shaking at 20-23° C. cAMP-GLO detection solution containing protein kinase A was added for 20 min at 20-23° C., followed by the addition of Kinase-Glo Reagent for 10 min. Each solution was carefully transferred to a white, opaque, 96-well plate and Luminescence was measured using a Victor Plate Reader (Perkin-Elmer). SAP97 knockdown experiments were additionally performed using a BRET-based biosensor (EPAC) for cAMP and the protocol was adapted from Barak et al. (2008) Mol. Pharmacol. 74, 585-594.

ERK Phosphorylation:

Following transfection, HEK 293 cells were re-seeded into 6-well plates. Cells were serum-starved for 1 hour at 37° C. in HBSS and then stimulated with 500 nM CRF agonist for the duration of the described time-points. Cells were lysed with lysis buffer (50 mM Tris, pH 8.0, 150 mM NaCl, and 0.1% Triton X-100) containing protease inhibitors (1 mM AEBSF, 10 μg/ml leupeptin, and 5 μg/ml aprotinin) for 20 min on a rocking platform at 4° C. Samples were collected into 1.5 mL Eppendorf tubes and centrifuged at 15,000 g for 15 min at 4° C. to pellet insoluble material. A Bronsted-Lowry protein assay was performed and 50 μg of protein was incubated overnight at room temperature in 3×SDS Loading Buffer containing 2-mercaptoethanol. Samples were separated by SDS-PAGE, transferred to a nitrocellulose membrane, and immunoblotted for ERK1/2 (rabbit anti-p44/42 mitogen-activated protein kinase (MAPK), 1:1000), phospho-ERK1/2 (rabbit anti-phospho-p44/42 MAPK, 1:1000), SAP97 (mouse anti-SAP97, 1:1000), and HA-CRFR1 expression (mouse anti-HA, 1:1000), followed by a horseradish peroxidase-conjugated secondary anti-rabbit antibody (1:10,000) or anti-mouse antibody (1:10,000) where appropriate. Proteins were detected using chemiluminescence with the enhanced chemiluminescence kit from GE Healthcare.

Statistical Analysis:

Densitometric data were normalized first for protein expression and the maximum value was set to 100, with all other values displayed as a percentage thereof. One-way analysis of variance test (ANOVA) was performed to determine significance, followed by a post-hoc Tukey multiple comparison test or Bonferroni's multiple comparisons test to determine which means were significantly different (p<0.05) from one another.

### Results

Proteomic Analysis of CRFR1-Interacting PDZ Proteins:

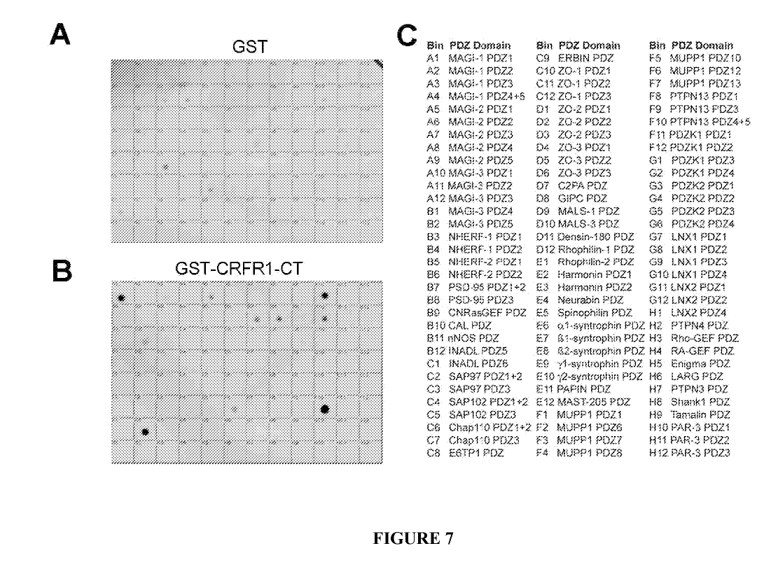

Array of 96 class I PDZ domains spotted on a gridded nylon membrane, as described in Fam et al. (2005). Proc. Natl. Acad. Sci. U.S.A. 102, 8042-8047; and He et al. (2006) J. Biol. Chem. 281, 2820-2827, were used to identify potential CRFR1 interacting PDZ domain-containing proteins. The PDZ array was overlaid with either purified glutathione-S-transferase (GST)-CRFR1-carboxyl-terminal tail or GST (as a control) and as can be observed in FIG. 7, a subset of PDZ proteins on the array exhibited binding to the GST-CRFR1-carboxyl-terminal tail. Specifically, the CRFR1 carboxyl-terminal tail selectively bound to a discrete group of PDZ domain-containing proteins: MAGI-1 PDZ1, MAGI-2 PDZ1, MAGI-3 PDZ1, PSD95 PDZ 1&2, PSD95 PDZ3, CAL PDZ, SAP97 PDZ1&2, PTPN13 PDZ 4&5, PDZK2 PDZ1 and MUPP1 PDZ 12.

SAP97 is Co-Immunoprecipitated with CRFR1 in a PDZ-Binding Motif-Dependent Manner:

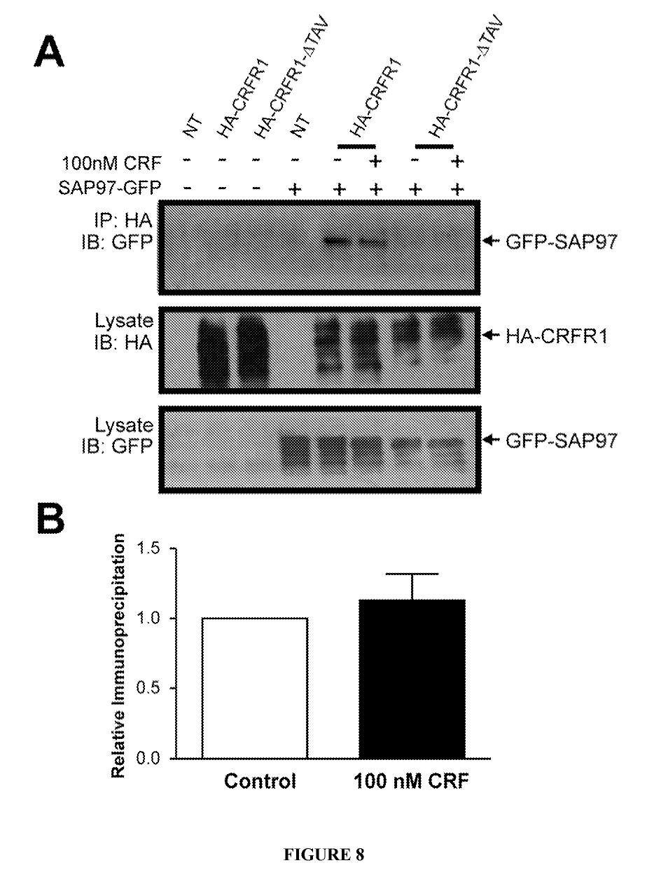

SAP97 was one of the candidate CRFR1 binding proteins identified in the proteomic PDZ domain screen (FIG. 7). (NOTE: can you provide the sequence of SAP97—human?) Therefore, it was confirmed that SAP97 interacts with CRFR1 by co-immunoprecipitation. GFP-SAP97 was co-immunoprecipitated with HA-CRFR1 from HEK 293 cells, but this interaction was not increased by agonist activation of HA-CRFR1 with 100 nM CRF (FIG. 8). This interaction was dependent upon an intact CRFR1 carboxyl-terminal PDZ binding motif, as the deletion of the last three critical amino acids (ΔTAV) of the CRFR1 carboxyl-terminal tail prevented the co-immunoprecipitation of SAP97 with the subsequent HA-CRFR1-ΔTAV mutant (FIG. 8). Thus, an intact CRFR1 carboxyl-terminal PDZ binding motif was required for SAP97 interactions with the receptor.

SAP97 Recruitment to the Plasma Membrane is CRFR1 PDZ Binding Motif-Dependent:

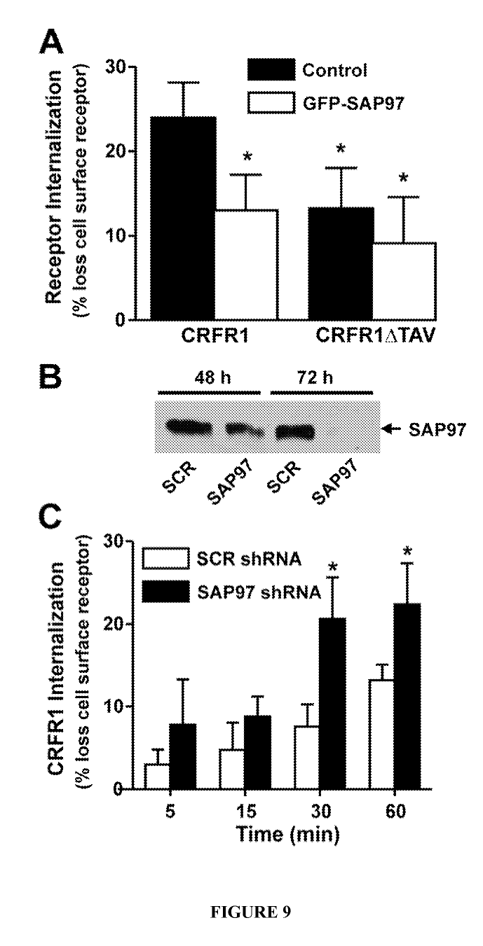

When expressed alone in HEK 293 cells GFP-SAP97 was diffusely localized throughout the cytoplasm and did not exhibit localization to the plasma membrane (data not shown). However, when GFP-SAP97 was co-expressed with HA-CRFR1, the GFP-SAP97 was predominantly localized with the receptor at the plasma membrane. When the CRFR1 PDZ motif was deleted from the carboxyl-terminus of the receptor (ΔTAV) the resulting HA-CRFR1-ΔTAV mutant did not promote the plasma membrane localization of GFP-SAP97. Thus, this data in combination with the co-immunoprecipitation data indicated that SAP97 was a CRFR1 interacting protein and that this interaction was dependent upon the CRFR1 PDZ binding motif SAP97 antagonizes CRFR1 endocytosis in a PDZ motif-dependent manner: PDZ interactions have been reported to regulate the endocytosis and trafficking of several GPCRs. Therefore, the effect of overexpressing GFP-SAP97 on the endocytosis of the wild-type CRFR1 and the CRFR1 mutant lacking a PDZ binding motif (ΔTAV) were examined. In cells expressing only wild-type CRFR1, agonist treatment for 30 min with 500 nM CRF at 37° C. resulted in a 24±4% loss of cell surface HA-CRFR1 as measured by flow cytometry (FIG. 9A). However, co-expression of GFP-SAP97 led to a significant attenuation of HA-CRFR1 endocytosis (FIG. 9A). Unexpectedly, deletion of the CRFR1 PDZ binding motif resulted in a HA-CRFR1-ΔTAV mutant that was impaired in its endocytosis when compared with the internalization of the wild-type receptor (FIG. 9A). GFP-SAP97 overexpression did not further antagonize the internalization of the HA-CRFR1-ΔTAV mutant (FIG. 9A). To examine the role of endogenous SAP97 in the regulation of agonist-stimulated CRFR1 endocytosis in HEK 293 cells, the cells were transfected with either scrambled shRNA or a shRNA SAP97 construct that was previously shown to knockdown SAP97 expression and tested CRFR1 internalization. As shown in FIG. 9B, the SAP97 shRNA construct effectively knocked down the expression of endogenous SAP97 protein expression in HEK 293 cells 72 h post-transfection. Consequently, all subsequent shRNA experiments were performed 72 h after HEK 293 cell transfection. shRNA knockdown of SAP97 significantly increased the maximal extent of HA-CRFR1 endocytosis following 30 and 60 minutes of agonist treatment with 500 nM CRF (FIG. 9C). Thus, taken together these data indicated that SAP97 functions to antagonize agonist-stimulated internalization of CRFR1, but that the interaction of the CRFR1 PDZ binding motif was required for effective internalization of the receptor.

SAP97 Co-Localizes with CRFR1 During Receptor Endocytosis:

The overexpression of GFP-SAP97 antagonized HA-CRFR1 endocytosis, but did not completely block the internalization of the receptor. Therefore, it was examined whether or not internalized HA-CRFR1 was either internalized as a complex with GFP-SAP97 or was internalized independently of GFP-SAP97. To do this, HEK 293 cells were transfected with both HA-CRFR1 and GFP-SAP97 and the HA-CRFR1 was labeled with Alexa Fluor 633-conjugated monoclonal HA mouse antibody (1:1000 dilution) for 45 min on ice. Labeled cells were then live cell imaged by laser scanning confocal microscopy. Each cell was allowed to warm to 37° C. and was imaged prior to the addition of 500 nM CRF and then was consecutively imaged every 30 s for 20 min. It was found that prior to agonist treatment, Alexa Fluor 633-conjugated mouse monoclonal HA antibody-labeled CRFR1 was colocalized with GFP-SAP97 at the cell surface. However, upon CRF treatment limited internalization of HA-CRFR1 was observed 30 min after agonist stimulation and internalized HA-CRFR1 that was internalized was co-localized with GFP-SAP97 in endocytic vesicles. This indicated that the pool of CRFR1 that could be endocytosed redistributed GFP-SAP97 into the endosomal compartment, despite the role for SAP97 in antagonizing CRFR1 endocytosis.

SAP97 does not Regulate CRFR1-Mediated cAMP Signaling:

Because SAP97overexpression antagonized CRFR1 internalization and SAP97 down-regulation enhanced CRFR1 endocytosis, it was determined whether or not either SAP97 or the CRFR1 PDZ binding motif contributed to the regulation of CRFR1-mediated cAMP formation. In cells transfected with HA-CRFR1 with and without GFP-SAP97 and treated with increasing doses of CRF, there was no significant change in the maximum efficacy for CRF-stimulated cAMP formation (FIG. 10A). Similarly, deletion of the CRFR1 PDZ binding motif had no effect on the maximum efficacy for CRF-stimulated cAMP formation in response to the activation of either the wild-type CRFR1 or the CRFR1-ΔTAV mutant (FIG. 10B). Consistent with what was observed following GFP-SAP97 overexpression, SAP97 shRNA knockdown did not result in an increase in the maximum efficacy for CRF-stimulated cAMP formation by the CRFR1 (FIG. 10C). Thus, SAP97 did not appear to contribute to the regulation of CRFR1 G protein-coupling.

CRFR1-Mediated ERK1/2 Phosphorylation is Dependent on Endogenous SAP97 Expression:

It was then examined whether endogenous SAP97 expression was required for CRFR1-mediated ERK1/2 phosphorylation. HEK293 cells were transiently transfected with and without HA-CRFR1 along with either scrambled shRNA or SAP97 shRNA to knockdown SAP97 expression. ERK1/2 phosphorylation in response to 500 nM CRF for 0, 2, 5, 15 and 30 min was determined by densitometric analysis of immunoblots. The treatment of non-transfected HEK 293 cells with 500 nM CRF led to an increase in detectable ERK1/2 phosphorylation at 5 min, which was likely due to endogenous CRFR2 that is expressed in these cells. However, in cells transfected with HA-CRFR1 and scrambled shRNA, 500 nM CRF treatment resulted in a more robust and sustained activation of ERK1/2 phosphorylation. shRNA knockdown of SAP97 protein expression led to attenuated CRFR1-mediated ERK1/2 phosphorylation following 500 nM CRF treatment, to levels that were comparable to those observed in non-transfected cells. The over-expression of GFP-SAP97 had no significant effect on ERK1/2 phosphorylation (data not shown). Taken together, these data suggested that SAP97 plays a direct role in regulating the activation of ERK1/2 signaling by the CRFR1, without modulating G protein coupling.

### Discussion

A subset of Class I PDZ proteins, including SAP97, have been found to interact with the CRFR1 C-tail on a proteomic PDZ domain array. SAP97 interacts with the carboxyl-terminal CRFR1 PDZ binding motif resulting in the recruitment of SAP97 to the cell surface. SAP97 functions to antagonize CRFR1 endocytosis, couples the receptor to the activation of the ERK1/2 signaling pathway, but does not alter CRFR1-mediated G protein coupling.

This is the first evidence for SAP97 as a regulator of CRFR1 trafficking and signaling. Specifically, SAP97 interacts with the CRFR1 PDZ binding motif to antagonize CRFR1 endocytosis and couples the receptor to the activation of ERK1/2 signaling without affecting G protein coupling.

The relevant portions of all references referred to herein are incorporated herein by reference.

1. A method of desensitizing 5-HT₂R signaling in a mammal comprising one or more of:
1) inhibiting, or at least reducing, CRFR1 activation of 5-HT₂R signaling;
2) blocking PDZ binding motifs in the carboxyl-terminal tail domains of at least one of CRFR1, 5-HT_{2A}R or 5-HT_{2C}R;
3) blocking the interaction of a 5-HT₂R with a PDZ-domain-containing protein; and
4) blocking the interaction of a CRFR1 with a PDZ-domain-containing protein.

2. The method of claim 1, wherein CRFR1 activation of 5-HT₂R signaling is inhibited by inhibiting trafficking of intracellular vesicles.

3. The method of claim 2, wherein an inhibitor of endocytosis is administered to the mammal.

4. The method of claim 3, wherein the inhibitor is monensin.

5. The method of claim 1, wherein an agent that blocks recycling of 5-HT_{2A}R to the cell surface is administered to the mammal.

6. The method of claim 5, wherein the agent is a dominant-negative Rab GTPase.

7. The method of claim 1, wherein the interaction of a 5-HT₂R or CRFR1 with PDZ-domain-containing protein is blocked.

8. The method of claim 7, wherein the PDZ-domain-containing protein is selected from the group consisting of MAGI-1 PDZ1, MAGI-2 PDZ1, MAGI-3 PDZ1, PSD95 PDZ 1&2, PSD95 PDZ3, CAL PDZ, SAP97 PDZ 1&2, PTPN13 PDZ 4&5, PDZK2 PDZ1, MPP3 PDZ, ERBIN PDZ and MUPP1 PDZ 12.

9. The method of claim 7, wherein the interaction is blocked by a nucleic acid or peptide inhibitor of a PDZ domain-containing protein.

10. The method of claim 1, wherein a peptide that blocks the CRFR1 or 5HT₂R PDZ binding motif is administered to the mammal.

11. The method of claim 10, wherein the blocking peptide comprises an amino acid sequence selected from the group consisting of FHSIKQSTAV, TRVSFHSIKQSTAV, and functionally equivalent derivatives thereof.

12. The method of claim 10, wherein the blocking peptide comprises an amino acid sequence selected from the group consisting of KDNSDGVNEKVSCV, and functionally equivalent derivatives thereof.

13. A method of screening candidate compounds for use to desensitize 5-HT₂R signaling comprising:
i) incubating the candidate compound with a CRFR1 or 5HT₂R-expressing cell line in the presence of SAP97; and
ii) determining the effect of the compound on the interaction of SAP97 with CRFR1 or 5HT₂R, wherein a determination of a difference in the interaction in comparison to a control interaction achieved by incubating a CRFR1 or 5HT₂R-expressing cell line in the presence of SAP97 with no compound indicates that the compound may desensitize 5-HT₂R signaling.

14. The method of claim 10, wherein a difference in activation of ERK1/2 signaling by CRFR1 is determined.

15. The method of claim 10, wherein a difference in SAP97 binding to the PDZ binding motif of CRFR1 or 5HT₂R is determined.

16. The method of claim 10, wherein a difference in receptor endocytosis is determined.


SEQUENCE LISTING (8 sequences)

SEQ ID NO: 1 - protein, 4 aa, Artificial Sequence
  blocking peptide
STAV

SEQ ID NO: 2 - protein, 10 aa, artificial sequence
  blocking peptide
FHSIKQSTAV

SEQ ID NO: 3 - protein, 14 aa, artificial sequence
  blocking peptide
TRVSFHSIKQSTAV

SEQ ID NO: 4 - protein, 14 aa, artificial sequence
  blocking peptide
KDNSDGVNEKVSCV

SEQ ID NO: 5 - protein, 444 aa, homosapiens
MGGHPQLRLVKALLLLGLNPVSASLQDQHCESLSLASNISGLQCNASVDLIGTCWPRSPAGQLVVRPCPAFFYGVRYNTTNNGYRECLANGSWAARVNYSECQEILNEEKKSKVHYHVAVIINYLGHCISLVALLVAFVLFLRLRPGCTHWGDQADGALEVGAPWSGAPFQVRRSIRCLRNIIHWNLISAFILRNATWFVVQLTMSPEVHQSNVGWCRLVTAAYNYFHVTNFFWMFGEGCYLHTAIVLTYSTDRLRKWMFICIGWGVPFPIIVAWAIGKLYYDNEKCWFGKRPGVYTDYIYQGPMILVLLINFIFLFNIVRILMTKLRASTTSETIQYRKAVKATLVLLPLLGITYMLFFVNPGEDEVSRVVFIYFNSFLESFQGFFVSVFYCFLNSEVRSAIRKRWHRWQDKHSIRARVARAMSIPTSPTRVSFHSIKQSTAV

SEQ ID NO: 6 - protein, 471 aa, homosapiens
MDILCEENTSLSSTTNSLMQLNDDTRLYSNDFNSGEANTSDAFNWTVDSENRTNLSCEGCLSPSCLSLLHLQEKNWSALLTAVVIILTIAGNILVIMAVSLEKKLQNATNYFLMSLAIADMLLGFLVMPVSMLTILYGYRWPLPSKLCAVWIYLDVLFSTASIMHLCAISLDRYVAIQNPIHHSRFNSRTKAFLKIIAVWTISVGISMPIPVFGLQDDSKVFKEGSCLLADDNFVLIGSFVSFFIPLTIMVITYFLTIKSLQKEATLCVSDLGTRAKLASFSFLPQSSLSSEKLFQRSIHREPGSYTGRRTMQSISNEQKACKVLGIVFFLFVVMWCPFFITNIMAVICKESCNEDVIGALLNVFVWIGYLSSAVNPLVYTLFNKTYRSAFSRYIQCQYKENKKPLQLILVNTIPALAYKSSQLQMGQKKNSKQDAKTTDNDCSMVALGKQHSEEASKDNSDGVNEKVSCV

SEQ ID NO: 7 - protein, 458 aa, homosapiens
MVNLRNAVHSFLVHLIGLLVWQCDISVSPVAAIVTDIFNTSDGGRFKFPDGVQNWPALSIVIIIIMTIGGNILVIMAVSMEKKLHNATNYFLMSLAIADMLVGLLVMPLSLLAILYDYVWPLPRYLCPVWISLDVLFSTASIMHLCAISLDRYVAIRNPIEHSRFNSRTKAIMKIAIVWAISIGVSVPIPVIGLRDEEKVFVNNTTCVLNDPNFVLIGSFVAFFIPLTIMVITYCLTIYVLRRQALMLLHGHTEEPPGLSLDFLKCCKRNTAEEENSANPNQDQNARRRKKKERRPRGTMQAINNERKASKVLGIVFFVFLIMWCPFFITNILSVLCEKSCNQKLMEKLLNVFVWIGYVCSGINPLVYTLFNKIYRRAFSNYLRCNYKVEKKPPVRQIPRVAATALSGRELNVNIYRHTNEPVIEKASDNEPGIEMQVENLELPVNPSSVVSERISSV

SEQ ID NO: 8 - protein, 904 aa, homosapiens
MPVRKQDTQRALHLLEEYRSKLSQTEDRQLRSSIERVINIFQSNLFQALIDIQEFYEVTLLDNPKCIDRSKPSEPIQPVNTWEISSLPSSTVTSETLPSSLSPSVEKYRYQDEDTPPQEHISPQITNEVIGPELVHVSEKNLSEIENVHGFVSHSHISPIKPTEAVLPSPPTVPVIPVLPVPAENTVILPTIPQANPPPVLVNTDSLETPTYVNGTDADYEYEEITLERGNSGLGFSIAGGTDNPHIGDDSSIFITKIITGGAAAQDGRLRVNDCILRVNEVDVRDVTHSKAVEALKEAGSIVRLYVKRRKPVSEKIMEIKLIKGPKGLGFSIAGGVGNQHIPGDNSIYVTKIIEGGAAHKDGKLQIGDKLLAVNNVCLEEVTHEEAVTALKNTSDFVYLKVAKPTSMYMNDGYAPPDITNSSSQPVDNHVSPSSFLGQTPASPARYSPVSKAVLGDDEITREPRKVVLHRGSTGLGFNIVGGEDGEGIFISFILAGGPADLSGELRKGDRIISVNSVDLRAASHEQAAAALKNAGQAVTIVAQYRPEEYSRFEAKIHDLREQMMNSSISSGSGSLRTSQKRSLYVRALFDYDKTKDSGLPSQGLNFKFGDILHVINASDDEWWQARQVTPDGESDEVGVIPSKRRVEKKERARLKTVKFNSKTRDKGEIPDDMGSKGLKHVTSNASDSESSYRGQEEYVLSYEPVNQQEVNYTRPVIILGPMKDRINDDLISEFPDKFGSCVPHTTRPKRDYEVDGRDYHFVTSREQMEKDIQEHKFIEAGQYNNHLYGTSVQSVREVAEKGKHCILDVSGNAIKRLQIAQLYPISIFIKPKSMENIMEMNKRLTEEQARKTFERAMKLEQEFTEHFTAIVQGDTLEDIYNQVKQIIEEQSGSYIWVPAKEKL